(19) United States
(12) Patent Application Publication
(10) Pub. No.: US 2012/0052061 A1
(43) Pub. Date: Mar. 1, 2012

(54) THERAPIES FOR TREATING CANCER USING COMBINATIONS OF COX-2 INHIBITORS AND ANTI-HER2(ERBB2) ANTIBODIES OR COMBINATIONS OF COX-2 INHIBITORS AND HER2(ERBB2) RECEPTOR TYROSINE KINASE INHIBITORS

(75) Inventors: Thomas M. Estok, Carlsbad, CA (US); Sara L. Zaknoen, Carlsbad, CA (US); Robert K. Mansfield, Carlsbad, CA (US); Tracy Lawhon, Encinitas, CA (US)

(73) Assignee: Tragara Pharmaceuticals Inc., San Diego, CA (US)

(21) Appl. No.: 13/131,856

(22) PCT Filed: Sep. 23, 2008
(86) PCT No.: PCT/US08/77410

§ 371 Date: Nov. 15, 2011

Related U.S. Application Data
(60) Provisional application No. 60/974,727, filed on Sep. 24, 2007.
(60) Provisional application No. 60/990,893, filed on Nov. 28, 2007.
(60) Provisional application No. 61/044,407, filed on Apr. 11, 2008.

(51) Int. Cl. A61K 39/395; A61K 31/517; A61N 5/00; A61K 31/5685; A61K 35/12; A61P 35/00; A61K 31/402; A61K 31/7068

(52) U.S. Cl. 424/133.1; 514/427; 514/266.21; 514/266.2; 514/49; 514/171; 424/93.7; 600/1

(57) ABSTRACT

Described herein are compositions and methods for using these compositions in the treatment of cancer, tumors, and tumor-related disorders in a subject.

### CROSS-REFERENCE

This application claims the benefit of U.S. Provisional Application No. 60/974,727, filed Sep. 24, 2007, U.S. Provisional Application No. 60/990,893, filed Nov. 28, 2007, and U.S. Provisional Application No. 61/044,407, filed Apr. 11, 2008, each of which is incorporated herein by reference in its entirety.

### FIELD

The present invention relates to combination therapies and the use of such combinations for the treatment of cancer, tumors, and tumor-related disorders.

### BACKGROUND

Cancer, tumors, tumor-related disorders, and neoplastic disease states are serious and often times life-threatening conditions. These diseases and disorders, which are characterized by rapidly-proliferating cell growth, continue to be the subject of research efforts directed toward the identification of therapeutic agents which are effective in the treatment thereof. Such agents prolong the survival of the patient, inhibit the rapidly-proliferating cell growth associated with the neoplasm, or effect a regression of the neoplasm.

Generally, surgery and radiation therapy are the first modalities considered for the treatment of cancer that is considered locally confined, and offer the best prognosis. Chemotherapy treatment of certain cancers typically results in disappointing survival rates but still offer a survival benefit.

Trastuzumab targets the HER2 receptor which is highly expressed and occasionally mutated in various forms of cancer. Three methods of use have been approved. 1. Trastuzumab is indicated for use as part of a treatment regimen containing doxorubicin, cyclophosphamide, and paclitaxel for the adjuvant treatment of patients with HER2-overexpressing, node-positive breast cancer. 2. Trastuzumab as a single agent is indicated for the treatment of patients with metastatic breast cancer whose tumors overexpress the HER2 protein and who have received one or more chemotherapy regimens for their metastatic disease. 3. Trastuzumab, in combination with paclitaxel, is indicated for treatment of patients with metastatic breast cancer whose tumors overexpress the HER2 protein and who have not received chemotherapy for their metastatic disease. If patients fail to respond to a trastuzumab treatment, additional treatment options include treatment with the combination of lapatinib and capecitabine.

Despite trastuzumab's approval for the treatment of breast cancer, as with most therapeutic agents, side-effects result from its use. For example, common side effects occurring in patients taking trastuzumab, include, fever, nausea, vomiting, infusion reactions, diarrhea, infections, increased cough, headache, fatigue, dyspnea, rash, neutropenia, anemia, and myalgia. Adverse reactions requiring interruption or discontinuation of trastuzumab treatment include severe infusion reactions, CHF, and significant decline in left ventricular cardiac function.

Of greater concern, is the growing view that, while utilization of trastuzumab for the treatment of tumors may initially shrink the size of the tumor, the tumor may eventually enlarge in size, indicating, among other things, the development of resistance. Trastuzumab may be representative of the types of therapeutic agents being used for cancer treatment in that its use has an effect on cancer, but because of other factors, which are not entirely known, the tumor develops resistance and progresses.

What is needed, therefore, are combination therapies and/or methods of treatment for cancer which take advantage of the synergy found in a therapeutic combination that could increase the effectiveness of the agents and reduce and/or eliminate the side effects typically associated with conventional treatments.

### SUMMARY OF THE INVENTION

Provided herein are methods of treating cancer based on the administration of a combination therapy comprising a 1,2-diphenylpyrrole derivative (a COX-2 selective inhibitor) and an anti-HER2 antibody. Also provided herein is a method for treating a subject having a tumor, a tumor-related disorder, and/or cancer, comprising administering to the subject, a therapeutically effective amount of a combination comprising 2-(4-ethoxyphenyl)-4-methyl-1-(4-sulfamoylphenyl)-pyrrole and trastuzumab. The methods may further include treatments wherein the combination is supplemented with one or more therapeutic agents or therapies.

Also provided herein are methods of treating cancer based on the administration of a combination of a 1,2-diphenylpyrrole derivative (a COX-2 selective inhibitor) and an inhibitor of HER2 [ErbB2]. The methods may further include treatments wherein the combination is supplemented with one or more therapeutic agents or therapies. The 1,2-diphenylpyrrole derivative and the inhibitor of HER2 [ErbB2] may be provided in separate dosage forms or combined in one dosage form (e.g. a fixed dose).

### Combination of a COX-2 Inhibitor and Anti HER2 Antibody

The methods and therapies of the invention have shown superior results compared to combinations based on other COX-2 inhibitors. For example, combinations according to the invention based on a combination of 2-(4-ethoxyphenyl)-4-methyl-1-(4-sulfamoylphenyl)-pyrrole and trastuzumab have shown 100% increase in tumor growth delay compared to administration of trastuzumab alone. Combinations containing 2-(4-ethoxyphenyl)-4-methyl-1-(4-sulfamoylphenyl)-pyrrole at a dose from about 5 to about 25 mg/kg and trastuzumab have shown significant synergism effects. For example, a combination containing 2-(4-ethoxyphenyl)-4-methyl-1-(4-sulfamoylphenyl)-pyrrole at a dose of about 10 mg/kg and trastuzumab at a dose of 15 mg/kg increased tumor growth delay by 100% compared to administration of trastuzumab alone. On the other hand, a combination containing celecoxib and tratusumab showed no significant effect on tumor growth delay when compared to administration of trastuzumab alone.

1,2-Diphenylpyrrole derivatives described herein have the general formula:

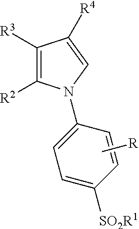

wherein:

R is a hydrogen atom, a halogen atom or an alkyl group having from 1 to 6 carbon atoms;

R¹ is an alkyl group having from 1 to 6 carbon atoms or an amino group;

R² is a phenyl group which is unsubstituted or is substituted by at least one substituent selected from the group consisting of substituents α and substituents β;

R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;

R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β; said aralkyl group are an alkyl group having from 1 to 6 carbon atoms and which are substituted by at least one aryl group as defined above;

said substituents α are selected from the group consisting of a hydroxy group, a halogen atom, an alkoxy group having from 1 to 6 carbon atoms and an alkylthio group having from 1 to 6 carbon atoms;

said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

In one embodiment, the invention provides a 1,2-diphenylpyrrole derivative having the formula:

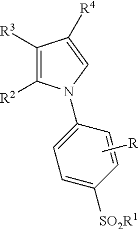

wherein:

R is a hydrogen atom, a halogen atom or an alkyl group having from 1 to 4 carbon atoms;

R¹ is a methyl group or an amino group;

R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;

R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

In one embodiment, the invention provides a 1,2-diphenylpyrrole derivative wherein:

R is a hydrogen atom;

R¹ is an amino group;

R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;

R³ is a hydrogen atom, a halogen atom, an alkyl group having from 1 to 4 carbon atoms or a haloalkyl group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

In one embodiment, R is a hydrogen atom. In another embodiment, R is a fluorine atom. In a further embodiment, R is a chlorine atom. In yet a further embodiment, R is a methyl group.

In one embodiment, R¹ is a methyl group. In another embodiment, R¹ is an amino group.

In one embodiment, R² is a phenyl group.

In one embodiment, R³ is a hydrogen atom. In another embodiment, R³ is a halogen atom.

In one embodiment, R⁴ is a hydrogen atom.

The term “aryl” refers to a carbocyclic aromatic hydrocarbon group having from 6 to 14 carbon atoms in one or more aromatic rings or such a group which is fused to a cycloalkyl group having from 3 to 10 carbon atoms, and the group is unsubstituted or it is substituted by at least one substituent selected from the group consisting of hydroxy groups, halogen atoms, lower alkoxy groups, lower alkylthio groups, lower alkyl groups, alkanoyloxy groups, mercapto groups, alknoylthio groups, lower alkylsulfinyl groups, lower alkyl groups having at least one substituent selected from the group consisting of cycloalkloxy groups, lower haloalkoxy groups, and lower alkylenedioxy groups.

In some embodiments, the 1,2-diphenylpyrrole derivative is selected from the group consisting of compounds 2-1-2-213 of Table 2 as disclosed in U.S. Pat. No. 6,887,893, which is herein incorporated in its entirety by reference.

In one embodiment, the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole. In another embodiment, the invention provides a method wherein the 1,2-diphenylpyrrole derivative is 2-(4-ethoxyphenyl)-4-methyl-1-(4-sulfamoylphenyl)-pyrrole.

In another embodiment, the 1,2-diphenylpyrrole derivative is 2-(4-ethoxyphenyl)-4-methyl-1-(4-sulfamoylphenyl)-pyrrole.

The methods for synthesizing 1,2-diphenylpyrrole derivatives, illustrated herein, are described in the Examples section and in U.S. RE39,420, which is incorporated herein by reference in its entirety.

In one embodiment the invention provides a combination therapy comprising a combination of a COX-2 selective inhibitor and a monoclonal antibody that selectively binds the HER2 receptor disclosed herein for the treatment and prevention of cancer, tumors, and tumor-related disorders, and neoplastic disease states.

In one embodiment, the monoclonal antibody that selectively binds the HER2 receptor is selected from trastuzumab, pertuzumab or trastuzumab-DM1. In another embodiment, the monoclonal antibody that selectively binds the HER2 receptor is trastuzumab.

### Methods of Use Based on a Combination of a Cox-2 Inhibitor and Anti HER2 Antibody

The invention provides a method for treating a subject having a tumor, a tumor-related disorder, and/or cancer, comprising administering to the subject, a therapeutically effective amount of a combination comprising 2-(4-ethoxyphenyl)-4-methyl-1-(4-sulfamoylphenyl)-pyrrole and trastuzumab.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the monoclonal antibody that selectively binds the HER2 receptor is trastuzumab.

In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative or the respective pharmaceutically acceptable salt, solvate or prodrug and a monoclonal antibody that selectively binds the HER2.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the 1,2-diphenylpyrrole derivative is selected from the group consisting of 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the 1,2-diphenylpyrrole derivative and the monoclonal antibody that selectively binds the HER2 receptor are administered sequentially in either order or simultaneously. In a further embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the 1,2-diphenylpyrrole derivative is administered first. In one embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the monoclonal antibody that selectively binds the HER2 receptor is administered first.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein administering the combination enhances treatment of the subject in comparison to a treatment of either a 1,2-diphenylpyrrole derivative or a monoclonal antibody that selectively binds the HER2 receptor alone. In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein administering the combination reduces the side effects of the treatment of tumors, tumor-related disorders, and/or cancer.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the cancer is breast cancer.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and a monoclonal antibody that selectively binds the HER2 receptor wherein the tumors or cancer is characterized by a cancer cell that overexpresses the HER2 receptor protein.

In one embodiment, the monoclonal antibody that selectively binds the HER2 receptor is an antibody selected from the group consisting of: trastuzumab, pertuzumab and trastuzumab-DM1.

In one embodiment the invention provides a method of inducing differentiation of tumor cells, the method comprising contacting the cells with an effective amount of a combination comprising a 1,2-diphenylpyrrole derivative and a monoclonal antibody that selectively binds the HER2 receptor whereby the combination induces differentiation of tumor cells.

In one embodiment, the invention provides a method of inducing differentiation of tumor cells, the method comprising contacting the cells with an effective amount of a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In one embodiment the invention provides a method of inhibiting proliferation of cancer cells, the method comprising contacting a cancer cell with a combination comprising a 1,2-diphenylpyrrole derivative and a monoclonal antibody that selectively binds the HER2 receptor whereby the combination inhibits proliferation of cancer cells. In one embodiment, the invention provides a method of inhibiting proliferation of cancer cells, the method comprising contacting a cancer cell with a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In another embodiment the invention provides a method for reducing proliferation of cancer cells, the method comprising delivering to the cells a combination comprising a 1,2-diphenylpyrrole derivative and a monoclonal antibody that selectively binds the HER2 receptor, whereby the reduction of cell proliferation is greater than a reduction caused by either a 1,2-diphenylpyrrole derivative alone or a monoclonal antibody that selectively binds the HER2 receptor alone.

In one embodiment, the invention provides a method for reducing proliferation of cancer cells, the method comprising delivering to the cells a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In a further embodiment the invention provides a method of inhibiting metastases of tumor cells, the method comprising administering an effective amount of a combination comprising a 1,2-diphenylpyrrole derivative and a monoclonal antibody that selectively binds the HER2 receptor such that the combination inhibits metastatic activity of tumor cells. In one embodiment, the invention provides a method of inhibiting metastases of tumor cells, the method comprising administering an effective amount of a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In one embodiment the invention provides a method for inducing apoptosis in cancer cells, the method comprising contacting the cancer cells with a combination comprising a 1,2-diphenylpyrrole derivative and a monoclonal antibody that selectively binds the HER2 receptor sufficient to induce apoptosis. In one embodiment, the invention provides a method for inducing apoptosis in cancer cells, the method comprising contacting the cancer cells with a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In another embodiment the invention provides a method for sensitizing cancer cells resistant to a monoclonal antibody that selectively binds the HER2 receptor to a monoclonal antibody that selectively binds the HER2 receptor, the method comprising administering a combination comprising a 1,2-diphenylpyrrole derivative and a monoclonal antibody that selectively binds the HER2 receptor wherein the combination sensitizes the cancer cells to the monoclonal antibody that selectively binds the HER2 receptor. In one embodiment, the invention provides a method for sensitizing cancer cells resistant to a monoclonal antibody that selectively binds the HER2 receptor to a monoclonal antibody that selectively binds the HER2 receptor, the method comprising administering a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In a further embodiment the invention provides a method of modulating prostaglandin synthesis in a cancer cell, the method comprising contacting the cell with a combination comprising a 1,2-diphenylpyrrole derivative and a monoclonal antibody that selectively binds the HER2 receptor wherein the combination inhibits prostaglandin synthesis in a cancer cell. In one embodiment, the invention provides a method of modulating prostaglandin synthesis in a cancer cell, the method comprising contacting the cell with a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In one embodiment the invention provides a method of modulating cyclooxygenase expression in a cancer cell, the method comprising delivering to the cell a combination comprising a 1,2-diphenylpyrrole derivative and a monoclonal antibody that selectively binds the HER2 receptor wherein the combination inhibits cyclooxygenase expression in a cancer cell. In one embodiment, the invention provides a method of modulating cyclooxygenase expression in a cancer cell, the method comprising delivering to the cell a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In one embodiment the invention provides a method of modulating angiogenesis in a cancer cell, the method comprising contacting the cell with a combination comprising a 1,2-diphenylpyrrole derivative and a monoclonal antibody that selectively binds the HER2 receptor wherein the combination inhibits angiogenesis in a cancer cell. In one embodiment the invention provides a method of modulating angiogenesis in a cancer cell, the method comprising contacting the cell with a combination comprising a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab. In another embodiment the invention provides a method of reducing the dosage in conventional treatment for neoplasia and/or neoplasia related disorders in a subject, the method comprising administering to a subject a combination of a 1,2-diphenylpyrrole derivative and a monoclonal antibody that selectively binds the HER2 receptor wherein the combination reduces the dosage in conventional treatment for neoplasia and/or neoplasia-related disorders. In one embodiment, the invention provides a method of reducing the dosage in conventional treatment for neoplasia and/or neoplasia related disorders in a subject, the method comprising administering to a subject a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In one embodiment the invention provides a method of treating neoplasia and/or neoplasia related disorders, the method comprising administering a combination of a 1,2-diphenylpyrrole derivative and a monoclonal antibody that selectively binds the HER2 receptor. In one embodiment, the invention provides a method of treating neoplasia and/or neoplasia related disorders, the method comprising administering a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

### Combination Therapy Based on a Combination of a Cox-2 Inhibitor and Anti HER2 Antibody

In some embodiments, the combination therapy comprising a combination of a 1,2-diphenylpyrrole derivative and a monoclonal antibody that selectively binds the HER2 receptor described herein, has an effect that is additive of the effects of the 1,2-diphenylpyrrole derivative alone and the effects of the monoclonal antibody that selectively binds the HER2 receptor alone. In another embodiment, the invention provides a combination therapy comprising, a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is additive of the effects of the 2-(4-ethoxyphenyl)-4-methyl-1-(4-sulfamoylphenyl)-pyrrole alone and the effects of trastuzumab alone.

In some other embodiments, the combination therapy comprising a combination of a 1,2-diphenylpyrrole derivative and a monoclonal antibody that selectively binds the HER2 receptor described herein, has an effect that is greater than the additive effects of the 1,2-diphenylpyrrole derivative alone and the effects of the monoclonal antibody that selectively binds the HER2 receptor alone. In another embodiment, the invention provides a combination therapy comprising, a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is greater than the additive effects of the 2-(4-ethoxyphenyl)-4-methyl-1-(4-sulfamoylphenyl)-pyrrole alone and the effects of trastuzumab alone.

In some embodiments, the combination therapy comprising a combination of a 1,2-diphenylpyrrole derivative and a monoclonal antibody that selectively binds the HER2 receptor described herein, has an effect that is greater than the effects of the 1,2-diphenylpyrrole derivative alone (e.g., cyclooxygenase-2 inhibition alone). In another embodiment, the invention provides a combination therapy comprising, a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is greater than the effects of the 2-(4-ethoxyphenyl)-4-methyl-1-(4-sulfamoylphenyl)-pyrrole alone.

In other embodiments, the combination therapy comprising a combination of a 1,2-diphenylpyrrole derivative and a monoclonal antibody that selectively binds the HER2 receptor described herein, has an effect that is greater than the effects of the monoclonal antibody that selectively binds the HER2 receptor alone. In another embodiment, the invention provides a combination therapy comprising, a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is greater than the effects of trastuzumab alone.

In other embodiments, the invention provides a method for treating cancer, tumors, and tumor-related disorders comprising administering a combination therapy comprising a combination of a 1,2-diphenylpyrrole derivative and a monoclonal antibody that selectively binds the HER2 receptor described herein, wherein the combination has an effect that is additive of the effects of the 1,2-diphenylpyrrole derivative alone and the effects of the monoclonal antibody that selectively binds the HER2 receptor alone. In further embodiments, the invention provides a method for treating cancer, tumors, and tumor-related disorders comprising administering a combination therapy comprising a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is additive of the effects of 2-(4-ethoxyphenyl)-4-methyl-1-(4-sulfamoylphenyl)-pyrrole alone and the effects of trastuzumab alone.

In some other embodiments, the invention provides a method for treating cancer, tumors, and tumor-related disorders, comprising administering a combination therapy comprising a combination of a 1,2-diphenylpyrrole derivative and a monoclonal antibody that selectively binds the HER2 receptor described herein, wherein the combination has an effect that is greater than the additive effects of the 1,2-diphenylpyrrole derivative alone and the effects of the monoclonal antibody that selectively binds the HER2 receptor alone. In other embodiments, the invention provides method for treating cancer, tumors, and tumor-related disorders, comprising administering a combination therapy comprising a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is greater than the additive effects of 2-(4-ethoxyphenyl)-4-methyl-1-(4-sulfamoylphenyl)-pyrrole alone and the effects of trastuzumab alone.

In some embodiments, the invention provides a method for treating cancer, tumors, and tumor-related disorders comprising administering a combination therapy comprising a combination of a 1,2-diphenylpyrrole derivative and a monoclonal antibody that selectively binds the HER2 receptor described herein, wherein the combination has an effect that is greater than the effects of the 1,2-diphenylpyrrole derivative alone (e.g., cyclooxygenase-2 inhibition alone). In other embodiments, the invention provides a method for treating cancer, tumors, and tumor-related disorders comprising administering a combination therapy comprising a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is greater than the effects of 2-(4-ethoxyphenyl)-4-methyl-1-(4-sulfamoylphenyl)-pyrrole alone.

In further embodiments, the invention provides a method for treating cancer, tumors, and tumor-related disorders comprising administering a combination therapy comprising a combination of a 1,2-diphenylpyrrole derivative and a monoclonal antibody that selectively binds the HER2 receptor described herein, wherein the combination has an effect that is greater than the effects of the monoclonal antibody that selectively binds the HER2 receptor alone.

In other embodiments, the invention provides a method for treating cancer, tumors, and tumor-related disorders comprising administering a combination therapy comprising a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab, wherein the combination has an effect that is greater than the effects of trastuzumab alone.

Synergism of the combination therapy comprising a combination of a 1,2-diphenylpyrrole derivative and a monoclonal antibody that selectively binds the HER2 receptor, may be used to obtain the desired effect at doses to which side effects are minimal. For example, a patient may be treated for a disease, disorder, or condition which benefits from HER2 receptor blockade, such as tumors, tumor-related diseases, cancer, neoplasia, while concomitantly being treated for a side effect of the HER2 receptor blockade, such as inflammation, through the benefit of the 1,2-diphenylpyrrole derivative inhibitor. In one embodiment, the invention provides a combination of 2-(4-ethoxyphenyl)-4-methyl-1-(4-sulfamoylphenyl)-pyrrole and trastuzumab which may be used to obtain the desired effect at doses to which side effects are minimal.

In one embodiment the invention provides a method for treating a subject having a cancer resistant to a monoclonal antibody that selectively binds HER2 comprising administering to the subject a therapeutically effective amount of a combination therapy comprising a 1,2-diphenylpyrrole derivative in combination with a monoclonal antibody that selectively binds the HER2 receptor. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab.

In one embodiment the invention provides a method for sensitizing cancer cells resistant to a monoclonal antibody that selectively binds the HER2 to a monoclonal antibody that selectively binds the HER2 receptor, the method comprising administering a combination comprising a 1,2-diphenylpyrrole derivative and a monoclonal antibody that selectively binds the HER2 receptor wherein the combination sensitizes the cancer cells to the monoclonal antibody that selectively binds the HER2 receptor. In one embodiment, the invention provides a method for sensitizing cancer cells resistant to a monoclonal antibody that selectively binds the HER2 to trastuzumab, the method comprising administering a combination comprising a 2-(4-ethoxyphenyl)-4-methyl-1-(4-sulfamoylphenyl)-pyrrole and trastuzumab wherein the combination sensitizes the cancer cells trastuzumab.

### Combination of a COX-2 Inhibitor and an Inhibitor of HER2 [ErbB2]

As indicated above, also provided herein are methods of treating cancer based on the administration of a combination of a 1,2-diphenylpyrrole derivative and an inhibitor of HER2 [ErbB2]. The methods may further include treatments wherein the combination is supplemented with one or more therapeutic agents or therapies. The 1,2-diphenylpyrrole derivative and the inhibitor of HER2 [ErbB2] may be provided in separate dosage forms or combined in one dosage form (e.g. a fixed dose).

In one embodiment, the invention provides a composition comprising a combination of a 1,2-diphenylpyrrole derivative and an inhibitor of HER2 [ErbB2] wherein the NSAID-induced side effects are substantially diminished. In another embodiment, the invention provides a composition comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577 and wherein the NSAID-induced side effects are substantially diminished.

### Methods of Use Based on a Combination of a Cox-2 Inhibitor and an Inhibitor of HER2 [ErbB2]

The invention provides a method for treating a subject having a tumor, a tumor-related disorder, and/or cancer, comprising administering to the subject, a therapeutically effective amount of a combination comprising 2-(4-ethoxyphenyl)-4-methyl-1-(4-sulfamoylphenyl)-pyrrole and an inhibitor of HER2 [ErbB2] selected from ARRY-380, CP-724714 or CP-654577.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the inhibitor of HER2 [ErbB2] is selected from ARRY-380, CP-724714 and CP-654577.

In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] or their respective pharmaceutically acceptable salt, solvate or prodrug.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the 1,2-diphenylpyrrole derivative and the inhibitor of HER2 [ErbB2] are administered sequentially in either order or simultaneously. In a further embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the 1,2-diphenylpyrrole derivative is administered first. In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the inhibitor of HER2 [ErbB2] is administered first. In another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein administering the combination enhances treatment of the subject in comparison to a treatment of either a 1,2-diphenylpyrrole derivative or an inhibitor of HER2 [ErbB2] alone. In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein administering the combination reduces the side effects of the treatment of tumors, tumor-related disorders, and/or cancer.

In one embodiment, the invention provides a method for treating a subject having a tumor, a tumor-related disorder, and/or cancer, wherein the tumor, tumor-related disorder, and/or cancer are characterized as overexpressing HER2/neu.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the cancer is selected from breast cancer, ovarian cancer, endometrial cancer, prostate cancer, gastric cancer, salivary gland cancer, pancreatic cancer, colorectal cancer, non-small cell lung cancers, oral cancers, and cutaneous squamous cell carcinoma.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the cancer is selected from non-small cell lung cancer, pancreatic cancer, breast cancer, ovarian cancer, colorectal cancer, and head and neck cancer.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of HER2 [ErbB2] wherein the cancer is breast cancer.

In one embodiment, the invention provides a method for treating a subject having cancer wherein the cancer is a carcinoma, a tumor, a neoplasm, a lymphoma, a melanoma, a glioma, a sarcoma, and a blastoma.

In one embodiment, the invention provides a method for treating a subject having a carcinoma wherein the carcinoma is selected from the group consisting of: adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In one embodiment, the invention provides a method for treating a subject having a tumor wherein the tumor is selected from the group consisting of astrocytic tumors, malignant mesothelial tumors, ovarian germ cell tumor, supratentorial primitive neuroectodermal tumors, Wilm's tumor, pituitary tumors, extragonadal germ cell tumor, gastrinoma, germ cell tumors, gestational trophoblastic tumor, brain tumors, pineal and supratentorial primitive neuroectodermal tumors, pituitary tumor, somatostatin-secreting tumor, endodermal sinus tumor, carcinoids, central cerebral astrocytoma, glucagonoma, hepatic adenoma, insulinoma, medulloepithelioma, plasmacytoma, vipoma, and pheochromocytoma.

In one embodiment, the invention provides a method for treating a subject having a neoplasm wherein the neoplasm is selected from the group consisting of intaepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma.

In one embodiment, the invention provides a method for treating a subject having a lymphoma wherein the lymphome is selected from the group consisting of: nervous system lymphoma, AIDS-related lymphoma, cutaneous T-cell lymphoma, non-Hodgkin's lymphoma, lymphoma, and Waldenstrom's macroglobulinemia.

In one embodiment, the invention provides a method for treating a subject having a melanoma wherein the melaoma is selected from the group consisting of: acral lentiginous melanoma, superficial spreading melanoma, uveal melanoma, lentigo maligna melanomas, melanoma, intraocular melanoma, adenocarcinoma nodular melanoma, and hemangioma.

In one embodiment, the invention provides a method for treating a subject having a sarcoma wherein the srcoma is selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma.

In one embodiment, the invention provides a method for treating a subject having a glioma wherein the glioma is selected from the group consisting of glioma, brain stem glioma, and hypothalamic and visual pathway glioma.

In one embodiment, the invention provides a method for treating a subject having a blastoma wherein the blastoma is selected from the group consisting of: pulmonary blastoma, pleuropulmonary blastoma, retinoblastoma, neuroblastoma, medulloblastoma, glioblastoma, and hemangiblastomas.

In one embodiment the inhibitor of HER2 [ErbB2] is a small molecule compound. In another embodiment the inhibitor of HER2 [ErbB2] is a small molecule compound selected from the group consisting of CP-724714, ARRY-380 and CPCP-654577 or their pharmaceutically acceptable salts, solvates, or prodrugs.

In one embodiment the invention provides a method of inducing differentiation of tumor cells, the method comprising contacting the cells with an effective amount of a combination comprising a 1,2-diphenylpyrrole derivative and an inhibitor of HER2 [ErbB2] whereby the combination induces differentiation of tumor cells. In one embodiment, the invention provides a method of inducing differentiation of tumor cells, the method comprising contacting the cells with an effective amount of a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In one embodiment the invention provides a method of inhibiting proliferation of cancer cells, the method comprising contacting a cancer cell with a combination comprising a 1,2-diphenylpyrrole derivative and an inhibitor of HER2 [ErbB2] whereby the combination inhibits proliferation of cancer cells. In one embodiment, the invention provides a method of inhibiting proliferation of cancer cells, the method comprising contacting a cancer cell with a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In another embodiment the invention provides a method for reducing proliferation of cancer cells, the method comprising delivering to the cells a combination comprising a 1,2-diphenylpyrrole derivative and an inhibitor of HER2 [ErbB2], whereby the reduction of cell proliferation is greater than a reduction caused by either a 1,2-diphenylpyrrole derivative alone or an inhibitor of HER2 [ErbB2] alone. In one embodiment, the invention provides a method for reducing proliferation of cancer cells, the method comprising delivering to the cells a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In one embodiment the invention provides a method of modulating autophosphorylation with a molecule of ATP, the method comprising delivering to a cancer cell an effective amount of a combination comprising a 1,2-diphenylpyrrole derivative and inhibitor of HER2 [ErbB2] wherein the combination inhibits autophosphorylation with a molecule of ATP. In one embodiment, the invention provides a method of modulating autophosphorylation with a molecule of ATP, the method comprising delivering to a cancer cell an effective amount of a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In a further embodiment the invention provides a method of inhibiting metastases of tumor cells, the method comprising administering an effective amount of a combination comprising a 1,2-diphenylpyrrole derivative and an inhibitor of HER2 [ErbB2] such that the combination inhibits metastatic activity of tumor cells. In one embodiment, the invention provides a method of inhibiting metastases of tumor cells, the method comprising administering an effective amount of a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In one embodiment the invention provides a method for inducing apoptosis in cancer cells, the method comprising contacting the cancer cells with a combination comprising a 1,2-diphenylpyrrole derivative and an inhibitor of HER2 [ErbB2] sufficient to induce apoptosis. In one embodiment, the invention provides a method for inducing apoptosis in cancer cells, the method comprising contacting the cancer cells with a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In another embodiment the invention provides a method for sensitizing EGFR [ErbB1] inhibitor resistant cancer cells to an inhibitor of HER2 [ErbB2], the method comprising administering a combination comprising a 1,2-diphenylpyrrole derivative and an inhibitor of HER2 [ErbB2] wherein the combination sensitizes the cancer cells to the inhibitor of HER2 [ErbB2]. In another embodiment the invention provides a method for sensitizing HER2 [ErbB2] inhibitor resistant cancer cells to an inhibitor of HER2 [ErbB2], the method comprising administering a combination comprising a 1,2-diphenylpyrrole derivative and an inhibitor of HER2 [ErbB2] wherein the combination sensitizes the cancer cells to the inhibitor of HER2 [ErbB2]. In another embodiment the invention provides a method for sensitizing cancer cells resistant to an inhibitor of HER2 [ErbB2] to an inhibitor of HER2 [ErbB2], the method comprising administering a combination comprising a 1,2-diphenylpyrrole derivative and an inhibitor of HER2 [ErbB2] wherein the combination sensitizes the cancer cells to the inhibitor of HER2 [ErbB2]. In one embodiment, the invention provides a method for sensitizing EGFR [ErbB1] inhibitor resistant cancer cells to an inhibitor of HER2 [ErbB2], the method comprising administering a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577. In one embodiment, the invention provides a method for sensitizing HER2 [ErbB2] inhibitor resistant cancer cells to an inhibitor of HER2 [ErbB2], the method comprising administering a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577. In one embodiment, the invention provides a method for sensitizing cancer cells resistant to an inhibitor of HER2 [ErbB2] to an inhibitor of HER2 [ErbB2], the method comprising administering a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In a further embodiment the invention provides a method of modulating prostaglandin synthesis in a cancer cell, the method comprising contacting the cell with a combination comprising a 1,2-diphenylpyrrole derivative and an inhibitor of HER2 [ErbB2] wherein the combination inhibits prostaglandin synthesis in a cancer cell. In one embodiment, the invention provides a method of modulating prostaglandin synthesis in a cancer cell, the method comprising contacting the cell with a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In one embodiment the invention provides a method of modulating cyclooxygenase expression in a cancer cell, the method comprising delivering to the cell a combination comprising a 1,2-diphenylpyrrole derivative and an inhibitor of HER2 [ErbB2] wherein the combination inhibits cyclooxygenase expression in a cancer cell. In one embodiment, the invention provides a method of modulating cyclooxygenase expression in a cancer cell, the method comprising delivering to the cell a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In one embodiment the invention provides a method of modulating angiogenesis in a cancer cell, the method comprising contacting the cell with a combination comprising a 1,2-diphenylpyrrole derivative and an inhibitor of HER2 [ErbB2] wherein the combination inhibits angiogenesis in a cancer cell. In one embodiment the invention provides a method of modulating angiogenesis in a cancer cell, the method comprising contacting the cell with a combination comprising a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577. In another embodiment the invention provides a method of reducing the dosage in conventional treatment for neoplasia and/or neoplasia related disorders in a subject, the method comprising administering to a subject a combination of a 1,2-diphenylpyrrole derivative and an inhibitor of HER2 [ErbB2] wherein the combination reduces the dosage in conventional treatment for neoplasia and/or neoplasia-related disorders. In one embodiment, the invention provides a method of reducing the dosage in conventional treatment for neoplasia and/or neoplasia related disorders in a subject, the method comprising administering to a subject a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In one embodiment the invention provides a method of treating neoplasia and/or neoplasia related disorders, the method comprising administering a combination of a 1,2-diphenylpyrrole derivative and an inhibitor of HER2 [ErbB2]. In one embodiment, the invention provides a method of treating neoplasia and/or neoplasia related disorders, the method comprising administering a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

In one embodiment the invention provides a method of modulating the immune response, the method comprising administering a combination of a 1,2-diphenylpyrrole derivative and an inhibitor of HER2 [ErbB2]. In one embodiment, the invention provides a method of modulating the immune response, the method comprising administering a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577.

### Compositions Based on a Combination of a COX-2 Inhibitor and an Inhibitor of HER2 [ErbB2]

In some embodiments, the composition comprising a combination of a 1,2-diphenylpyrrole derivative and an inhibitor of HER2 [ErbB2] described herein, has an effect that is additive of the effects of the 1,2-diphenylpyrrole derivative alone and the effects of the inhibitor of HER2 [ErbB2] alone. In another embodiment, the invention provides a composition comprising, a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, wherein the combination has an effect that is additive of the effects of the 2-(4-ethoxyphenyl)-4-methyl-1-(4-sulfamoylphenyl)-pyrrole alone and the effects of The inhibitor of HER2 [ErbB2] alone.

In some other embodiments, the composition comprising a combination of a 1,2-diphenylpyrrole derivative and an inhibitor of HER2 [ErbB2] described herein, has an effect that is greater than the additive effects of the 1,2-diphenylpyrrole derivative alone and the effects of the inhibitor of HER2 [ErbB2] alone. In another embodiment, the invention provides a composition comprising, a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, wherein the combination has an effect that is greater than the additive effects of the 2-(4-ethoxyphenyl)-4-methyl-1-(4-sulfamoylphenyl)-pyrrole alone and the effects of The inhibitor of HER2 [ErbB2] alone.

In some embodiments, the composition comprising a combination of a 1,2-diphenylpyrrole derivative and an inhibitor of HER2 [ErbB2] described herein, has an effect that is greater than the effects of the 1,2-diphenylpyrrole derivative alone (e.g., cyclooxygenase-2 inhibition alone). In another embodiment, the invention provides a composition comprising, a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, wherein the combination has an effect that is greater than the effects of the 2-(4-ethoxyphenyl)-4-methyl-1-(4-sulfamoylphenyl)-pyrrole alone.

In other embodiments, the composition comprising a combination of a 1,2-diphenylpyrrole derivative and an inhibitor of HER2 [ErbB2] described herein, has an effect that is greater than the effects of the inhibitor of HER2 [ErbB2] alone. In another embodiment, the invention provides a composition comprising, a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, wherein the combination has an effect that is greater than the effects of the inhibitor of HER2 [ErbB2] alone.

In other embodiments, the invention provides a method for treating cancer, tumors, and tumor-related disorders comprising administering a composition comprising a combination of a 1,2-diphenylpyrrole derivative and an inhibitor of HER2 [ErbB2] described herein, wherein the combination has an effect that is additive of the effects of the 1,2-diphenylpyrrole derivative alone and the effects of the inhibitor of HER2 [ErbB2] alone. In further embodiments, the invention provides a method for treating cancer, tumors, and tumor-related disorders comprising administering a composition comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577, wherein the combination has an effect that is additive of the effects of 2-(4-ethoxyphenyl)-4-methyl-1-(4-sulfamoylphenyl)-pyrrole alone and the effects of The inhibitor of HER2 [ErbB2] alone.

In some other embodiments, the invention provides a method for treating cancer, tumors, and tumor-related disorders, comprising administering a composition comprising a combination of a 1,2-diphenylpyrrole derivative and an inhibitor of HER2 [ErbB2] described herein, wherein the combination has an effect that is greater than the additive effects of the effects of the 1,2-diphenylpyrrole derivative alone and the effects of the inhibitor of HER2 [ErbB2] alone. In other embodiments, the invention provides a method for treating cancer, tumors, and tumor-related disorders, comprising administering a composition comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, wherein the combination has an effect that is greater than the additive effects of 2-(4-ethoxyphenyl)-4-methyl-1-(4-sulfamoylphenyl)-pyrrole alone and the effects of The inhibitor of HER2 [ErbB2] alone.

In some embodiments, the invention provides a method for treating cancer, tumors, and tumor-related disorders comprising administering a composition comprising a combination of a 1,2-diphenylpyrrole derivative and an inhibitor of HER2 [ErbB2] described herein, wherein the combination has an effect that is greater than the effects of the 1,2-diphenylpyrrole derivative alone (e.g., cyclooxygenase-2 inhibition alone). In other embodiments, the invention provides a method for treating cancer, tumors, and tumor-related disorders comprising administering a composition comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 or CP-654577, wherein the combination has an effect that is greater than the effects of is 2-(4-ethoxyphenyl)-4-methyl-1-(4-sulfamoylphenyl)-pyrrole alone.

In further embodiments, the invention provides a method for treating cancer, tumors, and tumor-related disorders comprising administering a composition comprising a combination of a 1,2-diphenylpyrrole derivative and an inhibitor of HER2 [ErbB2] described herein, wherein the combination has an effect that is greater than the effects of the inhibitor of HER2 [ErbB2] alone. In other embodiments, the invention provides a method for treating cancer, a tumor or a tumor-related disorder comprising administering a composition comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, wherein the combination has an effect that is greater than the effects of the inhibitor of HER2 [ErbB2] alone.

Synergism of the composition comprising a combination of a 1,2-diphenylpyrrole derivative and an inhibitor of HER2 [ErbB2], may be used to obtain the desired effect at doses to which side effects are minimal. For example, a patient may be treated for a disease, disorder, or condition which benefits from HER2 [ErbB2] inhibition, such as tumors, tumor-related diseases, cancer, neoplasia, while concomitantly being treated for a side effect of the HER2 [ErbB2] inhibition, such as inflammation, through the benefit of the 1,2-diphenylpyrrole derivative inhibitor. In one embodiment, the invention provides a combination of 2-(4-ethoxyphenyl)-4-methyl-1-(4-sulfamoylphenyl)-pyrrole and an inhibitor of HER2 [ErbB2] selected from ARRY-380, CP-724714 and CP-654577 which may be used to obtain the desired effect at doses to which side effects are minimal.

The composition comprising a combination of a 1,2-diphenylpyrrole derivative and an inhibitor of HER2 [ErbB2] may be applied as a sole therapy or may involve one or more other materials and treatment agents.

Thus, the composition comprising a combination of a 1,2-diphenylpyrrole derivative and an EGFR [ErbB1] inhibitor, may be applied with one or more other anti-tumor substances, for example, those selected from, mitotic inhibitors, for example vinblastine; alkylating agents, for example, cis-platin, carboplatin, and cyclophosphamide; anti-metabolites, for example capecitabine, 5-fluorouracil, cytosine arabinoside and hydroxyurea, or, for example, anti-metabolites such as pemetrexed, methotrexate, raltitrexed, or N-(5-[N-(3,4-dihydro-2-methyl-4-oxoquinazolin-6-ylmethyl)-N-methylamino]-2-thenyl)-L-glutamic acid; growth factor inhibitors; cell cycle inhibitors; intercalating antibiotics, for example adriamycin and bleomycin; enzymes, for example interferon; aromatase inhibitors, for example letrozole, anastrozole and exemestane; monoclonal antibodies, for example trastuzumab, pertuzumab and trastuzumab-DM1; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide).

In one embodiment, the invention provides a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, may be applied with one or more other anti-tumor substances, for example, those selected from, mitotic inhibitors, for example vinblastine; alkylating agents, for example, cis-platin, carboplatin, and cyclophosphamide; anti-metabolites, for example capecitabine, 5-fluorouracil, cytosine arabinoside and hydroxyurea, or, for example, anti-metabolites such as pemetrexed, methotrexate, raltitrexed, or N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; aromatase inhibitors, for example letrozole, anastrozole or exemestane; monoclonal antibodies, for example trastuzumab, pertuzumab and trastuzumab-DM1; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide).

In one embodiment, the invention provides a method for inhibiting abnormal cell growth in a subject comprising administering to the subject an effective amount of a composition comprising a combination of a 1,2-diphenylpyrrole derivative and an inhibitor of HER2 [ErbB2], or pharmaceutically acceptable salt, solvate or prodrug thereof, in combination with radiation therapy effective in inhibiting abnormal cell growth in the subject. Techniques for administering radiation therapy are known to a person of skill in the art and these techniques can be used in the combination therapy described herein.

In one embodiment the invention provides a method for treating a subject having an EGFR [ErbB1] inhibitor resistant cancer cell comprising administering to the subject a therapeutically effective amount of a composition comprising a 1,2-diphenylpyrrole derivative in combination with an inhibitor of HER2 [ErbB2]. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577. In one embodiment the invention provides a method for treating a subject having a HER2 [ErbB2] inhibitor resistant cancer cell comprising administering to the subject a therapeutically effective amount of a composition comprising a 1,2-diphenylpyrrole derivative in combination with an inhibitor of HER2 [ErbB2]. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577. In one embodiment the invention provides a method for treating a subject having a cancer cell resistant to an inhibitor of HER2 [ErbB2] comprising administering to the subject a therapeutically effective amount of a composition comprising a 1,2-diphenylpyrrole derivative in combination with an inhibitor of HER2 [ErbB2]. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577.

Provided herein is a pharmaceutical composition for treating cancer comprising a combination of a 1,2-diphenylpyrrole derivative and an inhibitor of HER2 [ErbB2] and a pharmaceutically acceptable excipient or carrier.

In one embodiment, the invention provides a pharmaceutical composition comprising a combination of 2-(4-ethoxyphenyl)-4-methyl-1-(4-sulfamoylphenyl)-pyrrole and CP-724714 as an active ingredient, or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers.

In another embodiment, the 1,2-diphenylpyrrole derivative is 2-(4-ethoxyphenyl)-4-methyl-1-(4-sulfamoylphenyl)-pyrrole and a pharmaceutically acceptable excipient or carrier.

In another embodiment, the inhibitor of HER2 [ErbB2] is selected from ARRY-380, CP-724714 and CP-654577 and a pharmaceutically acceptable excipient or carrier. In a further embodiment the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577 and a pharmaceutically acceptable excipient or carrier.

In one embodiment, the invention provides a pharmaceutical composition for treating cancer comprising a combination of a 1,2-diphenylpyrrole derivative selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole. In another embodiment, the invention provides a method wherein the 1,2-diphenylpyrrole derivative is 2-(4-ethoxyphenyl)-4-methyl-1-(4-sulfamoylphenyl)-pyrrole and an inhibitor of HER2 [ErbB2] and a pharmaceutically acceptable excipient or carrier.

### INCORPORATION BY REFERENCE

All publications, patents, and patent applications described in this specification are herein incorporated by reference to the same extent as if each individual publication, patent, or patent application was specifically and individually indicated to be incorporated by reference.

### BRIEF DESCRIPTION OF THE DRAWINGS

FIG. 1 provides graphs illustrating COX-2 expression levels in colorectal cancer. The overall 10-year survival curves of patients with Cox-2 negative and Cox-2 positive are shown for the entire cohort, P=0.0006 (A), as well as for patients with stage I/II, P=0.0271 (B), or stage III, P=0.0081 (C) disease.

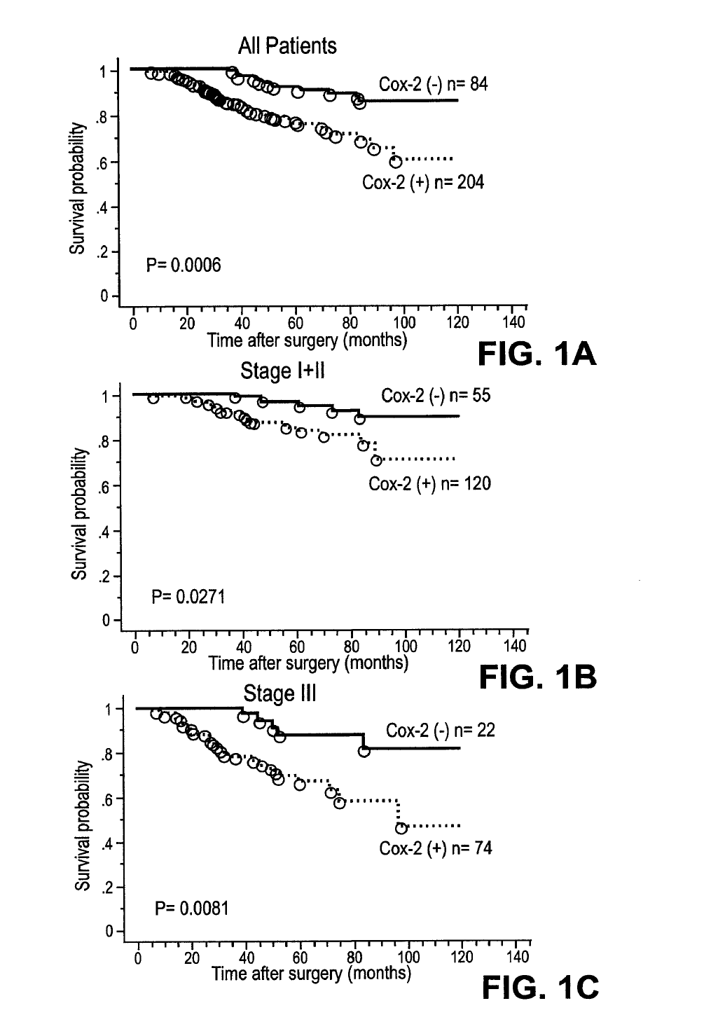

FIG. 2 provides a graph illustrating tumor growth delay in a BT474 xenograph experiment.

FIG. 3 provides a graph illustrating tumor growth delay in a MCF-7 xenograph experiment.

### DETAILED DESCRIPTION

Provided herein are methods of treating cancer based on the administration of a combination therapy comprising a 1,2-diphenylpyrrole derivative (a COX-2 selective inhibitor) and an anti-HER2 antibody. Also provided herein is a method for treating a subject having a tumor, a tumor-related disorder, and/or cancer, comprising administering to the subject, a therapeutically effective amount of a combination comprising 2-(4-ethoxyphenyl)-4-methyl-1-(4-sulfamoylphenyl)-pyrrole and trastuzumab. Additionally, methods are provided for treating cancer based on the administration of a combination therapy comprising a 1,2-diphenylpyrrole derivative (a COX-2 selective inhibitor) and a small molecule receptor tyrosine kinase inhibitor of HER2 [ErbB2]. Also provided herein is a method for treating a subject having a tumor, a tumor-related disorder, and/or cancer, comprising administering to the subject, a therapeutically effective amount of a combination comprising 2-(4-ethoxyphenyl)-4-methyl-1-(4-sulfamoylphenyl)-pyrrole and a small molecule receptor tyrosine kinase inhibitor of HER2 [ErbB2] selected from CP-724714, ARRY-380 and CP-654577.

The methods may further include treatments wherein the combination is supplemented with one or more therapeutic agents or therapies.

The methods and therapies of the invention have shown superior results compared to combinations based on other COX-2 inhibitors. For example, combinations according to the invention comprising 2-(4-ethoxyphenyl)-4-methyl-1-(4-sulfamoylphenyl)-pyrrole and trastuzumab have shown 100% increase in tumor growth delay compared to a combination including celecoxib and trastuzumab. Combinations containing 2-(4-ethoxyphenyl)-4-methyl-1-(4-sulfamoylphenyl)-pyrrole at a dose from about 5 to about 25 mg/kg and trastuzumab have shown significant synergism effects. For example, combinations containing 2-(4-ethoxyphenyl)-4-methyl-1-(4-sulfamoylphenyl)-pyrrole at a dose of about 10 mg/kg and trastuzumab at a dose of 15 mg/kg increased tumor growth delay by 100% compared to administration of trastuzumab alone. On the other hand, a combination containing celecoxib and tratusumab showed no significant effect on tumor growth delay when compared to administration of trastuzumab alone.

To facilitate understanding of the disclosure set forth herein, a number of terms are defined below.

As used herein, “abnormal cell growth,” refers to cell growth that is independent of normal regulatory mechanisms (e.g., loss of contact inhibition), including the abnormal growth of normal cells and the growth of abnormal cells.

“Neoplasia” as described herein, is an abnormal, unregulated and disorganized proliferation of cells that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. In some embodiments, the neoplasm can be benign or malignant.

“Metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

As discussed herein, “angiogenesis” is prominent in tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors such as renal cell carcinoma, hepatocellular carcinoma, and benign tumors such as acoustic neuroma, and neurofibroma. Angiogenesis has been associated with blood-born tumors such as leukemias. It is believed that angiogenesis plays a role in the abnormalities in the bone marrow that give rise to leukemia. Prevention of angiogenesis could halt the growth of cancerous tumors and the resultant damage to the subject due to the presence of the tumor.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

### Cyclooxygenase

Cyclooxygenase (COX) is an enzyme that is responsible for the formation of important biological mediators called prostanoids, including prostaglandins, prostacyclin and thromboxane. COX converts arachidonic acid, an ω-6 essential fatty acid, to prostaglandin H₂ (PGH₂), the precursor of the series-2 prostanoids. The enzyme contains two active sites: a heme with peroxidase activity, responsible for the reduction of PGG₂ to PGH₂, and a cyclooxygenase site, where arachidonic acid is converted into the hydroperoxy endoperoxide prostaglandin G₂ (PGG₂). The reaction proceeds through a hydrogen atom abstraction from arachidonic acid by a tyrosine radical generated by the peroxidase active site, then two oxygen molecules react with the arachidonic acid radical, giving PGG₂.

COX-1 is a constitutive enzyme responsible for biosynthesis of prostaglandins in the gastric mucosa and in the kidney among other sites. COX-2 is an enzyme that is produced by an inducible gene that is responsible for biosynthesis of prostaglandins in inflammatory cells. Inflammation causes induction of COX-2, leading to release of prostanoids (prostaglandin E2), which sensitize peripheral nociceptor terminals and produce localized pain hypersensitivity, inflammation and edema.

### Overexpression of COX-2 and Cancer

The overexpression of COX-2 and also the upstream and downstream enzymes of the prostaglandin synthesis pathway have been demonstrated in multiple cancer types and some pre-neoplastic lesions. Direct interactions of prostaglandins with their receptors through autocrine or paracrine pathways to enhance cellular survival or stimulate angiogenesis have been proposed as the molecular mechanisms underlying the pro-carcinogenic functions of COX enzymes.

Studies indicate that prostaglandins synthesized by cyclooxygenase play a role in the initiation and promotion of cancer. Aberrant COX-2 expression was first reported in colorectal carcinomas and adenomas, and has now been detected in various human cancers, including those of the breast. Moreover, COX-2 is overexpressed in neoplastic lesions of the colon, breast, lung, prostate, esophagus, pancreas, intestine, cervix, ovaries, urinary bladder and head and neck (see Table 1 below).

TABLE 1
COX-2 Expression in Tumors

| Tumor Type | % Tissue expressing COX-2 |
|---|---|
| Colorectal Cancer | 70-95 |
| Non-small Cell Lung Cancer | 70-90 |
| Gastric Cancer | 45-75 |
| Pancreatic Cancer | 40-80 |
| Glioblastoma Multiforme | 40-70 |
| Bladder Cancer | 50-60 |
| Esophageal Cancer | 50-60 |
| Breast Cancer | 40-50 |
| Ovarian Cancer | 40-60 |
| Prostate Cancer | 40-60 |

COX-2 overexpression in murine mammary glands is sufficient to cause tumor formation. In several in vitro and animal models, COX-2 inhibitors have inhibited tumor growth and metastasis.

In addition to cancers per se, COX-2 is also expressed in the angiogenic vasculature within and adjacent to hyperplastic and neoplastic lesions indicating that COX-2 plays a role in angiogenesis. In both the mouse and rat, COX-2 inhibitors markedly inhibited bFGF-induced neovascularization. The utility of COX-2 inhibitors as chemopreventive, antiangiogenic and chemotherapeutic agents is described in the literature (Koki et al., Exp. Opin., Invest. Drugs, 1999, 8(10) 1623-38).

Additionally, several studies have suggested that COX-2 expression is associated with parameters of aggressive breast cancer, including large tumor size, positive axillary lymph node metastases and HER2-positive tumor status. Studies of mammary tumors in mice and rats have indicated that moderate to high COX-2 expression is related to the genesis of mammary tumors that are sensitive to treatment with nonspecific and specific COX-2 inhibitors. Studies of the relationship between the HER2 TKR and COX-2 have shown a link between HER2 signaling and COX-2 expression in HER2-positive breast cancer (Subbaramaiah et al., J. Biol. Chem., 2002, 277, 18649-657).

### Receptor Tyrosine Kinases

Protein tyrosine kinases are a class of enzymes that catalyze the transfer of a phosphate group from ATP or GTP to the tyrosine residue located on protein substrates. Protein tyrosine kinases clearly play a role in normal cell growth. Many of the growth factor receptor proteins function as tyrosine kinases and it is by this process that they effect signaling. The interaction of growth factors with these receptors is a necessary event in normal regulation of cell growth. Under certain conditions, however, as a result of either mutation or overexpression, these receptors can become deregulated; the result of which is uncontrolled cell proliferation which can lead to tumor growth and ultimately to cancer (Wilks, Adv. Cancer Res., 1993, 60, 43). Among the growth factor receptor kinases and their proto-oncogenes that have been identified and which are targets of the combinations presented herein are the epidermal growth factor receptor kinase (EGFR kinase, the protein product of the erbB oncogene), and the product produced by the erbB-2 (also referred to as the neu or HER2) oncogene. Since the phosphorylation event is a necessary signal for cell division to occur and since overexpressed or mutated kinases have been associated with cancer, an inhibitor of this event, a protein tyrosine kinase inhibitor, will have therapeutic value for the treatment of cancer and other diseases characterized by uncontrolled or abnormal cell growth. For example, overexpression of the receptor kinase product of the erbB-2 oncogene has been associated with human breast and ovarian cancers (Slamon et. al., Science, 1989, 244, 707). Deregulation of EGFR kinase has been associated with epidermoid tumors and tumors involving other major organs. Because of the importance of the role played by deregulated receptor kinases in the pathogenesis of cancer, many recent studies have dealt with the development of specific protein tyrosine kinase inhibitors as potential anti-cancer therapeutic agents.

Receptor tyrosine kinases span the cell membrane and possess an extracellular binding domain for growth factors such as epidermal growth factor (EGF), a transmembrane domain, and an intracellular portion which functions as a kinase to phosphorylate specific tyrosine kinase residues in proteins and hence to influence cell proliferation. The EGF receptor tyrosine kinase family has four members: EGFR (HER1, erbB1); HER2(c-erbB2, erbB2, neu); HER3 (erbB3); and HER4 (erbB4). The ErbB receptors generally transduce signals through two pathways. It is known that such kinases are frequently and aberrantly expressed in common human cancers such as breast cancer, gastrointestinal cancer of colon, rectum or stomach, leukemia, and ovarian, bronchial or pancreatic cancer. As discussed previously, epidermal growth factor receptor (EGFR), is mutated and/or overexpressed in many human cancers such as brain, lung, squamous cell, bladder, gastric, breast, head and neck, oesophageal, gynecological and thyroid tumors.

### HER2

As indicated above, HER2 (also known as ErbB-2 or neu) is a member of the epidermal growth factor receptor (ErbB) family and is notable for its role in the pathogenesis of breast cancer and as a target of treatment. It is a cell membrane surface-bound receptor tyrosine kinase and is normally involved in the signal transduction pathways leading to cell growth and differentiation. HER2 is thought to be an orphan receptor, with none of the EGF family of ligands able to activate it. However, ErbB receptors dimerise on ligand binding, and HER2 is the preferential dimerisation partner of other members of the ErbB family.

The HER2 gene is a proto-oncogene located at the long arm of human chromosome 17(17q11.2-q12). Approximately 25-30 percent of breast cancers have an amplification of the HER2 gene or overexpression of its protein product. Overexpression of this receptor in breast cancer is associated with increased disease recurrence and worse prognosis. The oncogene neu is so-named because it was derived from a neuroglioblastoma cell line in rat. HER2 is named because it has similar structure to human epidermal growth factor receptor, or HER1. ErbB2 was named for its similarity to ErbB (avian erythroblastosis oncogene B), the oncogene later found to code for EGFR. Gene cloning showed that neu, HER2, and ErbB2 were the same.

In general, there are two methods of determining HER2 status. First, measurement of gene amplification: FISH or fluorescence in-situ hybridization is a gene-based diagnostic test used to identify amplified HER2 genes and therefore excess HER2 protein. If the test shows an excess number of genes, the test is considered HER2 positive. If the test shows a normal number of genes, the test is considered HER2 negative. Second, measurement of protein expression: IHC or immunohistochemistry, a protein-based diagnostic test used to identify overexpressed HER2 protein caused by too many copies of the HER2 gene. IHC measures HER2 protein overexpression on different levels: 0, 1+, 2+and 3+. If the test is 2+, it is recommends that a FISH test should be conducted to confirm HER2 positive or negative status. If the tumor is 3+, it is HER2 positive.

There is increasing evidence that cyclooxygenase-2 (COX-2) may mediate the effects of HER2. As discussed above, COX-2 catalyzes the conversion of arachidonic acid to prostaglandins (PGs). High levels of COX-2 and its main product, PGE2, have been found in human breast cancer cells and tumors that overexpress HER2 but not in normal breast tissue. It has been suggested that COX-2 overexpression increases resistance to apoptosis, particularly NO-mediated apoptosis. There also appears to be a link between Akt activity and COX-2 expression.

### Breast Cancer

Cancers associated with overexpression of HER2 include breast, ovarian, endometrial, prostate, gastric, salivary gland, pancreatic, colorectal, oral and non-small cell lung cancers. Breast cancer has been a focus of anti-HER2 treatments.

Today, among women in the United States, breast cancer remains the most frequent diagnosed cancer. One in 8 women in the United States is at risk of developing breast cancer in their lifetime. Age, family history, diet, and genetic factors have been identified as risk factors for breast cancer. Breast cancer is the second leading cause of death among women.

Available treatments for breast cancer include radiation therapy, chemotherapy, hormone therapy, antibody therapy or tyrosine kinase inhibitor therapy as adjuvant.

Radiation therapy is a cancer treatment that uses high-energy x-rays or other types of radiation to kill cancer cells or keep them from growing. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the spinal column, an organ, or a body cavity such as the abdomen, the drugs mainly affect cancer cells in those areas (regional chemotherapy). The way chemotherapy is given depends on the type and stage of the cancer being treated.

Different chemotherapeutic agents are known in art for treating breast cancer. Cytoxic agents used for treating breast cancer include doxorubicin, cyclophosphamide, methotrexate, 5-fluorouracil, mitomycin C, mitoxantrone, paclitaxel, taxane formulations such as by way of example only, Abraxane® (ABI-007), Paclitaxel-Cremophor EL, Paclitaxel poliglumex, and Paclitaxel injectable emulsion (PIE), gemcitabine, docetaxel, capecitabine and epirubicin.

Other chemotherapy against breast cancer includes treatment with one or more of bendamustine, carboplatin (for example, Paraplatin®), carmustine (for example, BCNU®), chlorambucil (for example, Leukeran®), cisplatin (for example, Platinol®), cyclophosphamide injection (for example, Cytoxan®), oral cyclophosphamide (for example, Cytoxan®), dacarbazine (for example, DTIC®), ifosfamide (for example, Ifex®), lomustine (for example, CCNU®), mechlorethamine (for example, nitrogen mustard, Mustargen®), melphalan (for example, Alkeran®), procarbazine (for example, Matulane®), bleomycin (for example, Blenoxane®), doxorubicin (for example, Adriamycin®, Rubex®), epirubicin, Idarubicin (for example, Idamycin®), mitoxantrone (for example, Novantrone®), gemcitabine (for example, Gemzar®), oral mercaptopurine (for example, Purinethol®), methotrexate, pentostatin IV (for example, Nipent®), oral thioguanine (for example, Lanvis®), oral etoposide (for example, VP-16, VePesid®, Etopophos)-etoposide IV (for example, VP-16, VePesid®, Etopophos), vinblastine (for example, Velban®), vincristine (for example, Oncovin®), vinorelbine (for example, Navelbine®), dexamethasone (for example, Decadron®), methylprednisolone (for example, Medrol®), and prednisone (for example, Deltasone®). The present disclosure contemplates adding one or more of these chemotherapeutic agents to the therapies disclosed herein, for example, a combination therapy comprising 2-(4-ethoxyphenyl)-4-methyl-1-(4-sulfamoylphenyl)-pyrrole and trastuzumab.

Hormone therapy is a cancer treatment that removes hormones or blocks their action and stops cancer cells from growing. Hormone therapy with tamoxifen is often given to patients with early stages of breast cancer and those with metastatic breast cancer (cancer that has spread to other parts of the body). Hormone therapy with tamoxifen or estrogens can act on cells all over the body and may increase the chance of developing endometrial cancer. Hormone therapy with an aromatase inhibitor is given to some postmenopausal women who have hormone-dependent breast cancer. Hormone-dependent breast cancer needs the hormone estrogen to grow.

Aromatase inhibitors decrease the body's estrogen by blocking an enzyme called aromatase from turning androgen into estrogen. For the treatment of early stage breast cancer, certain aromatase inhibitors may be used as adjuvant therapy instead of tamoxifen or after 2 or more years of tamoxifen. For the treatment of metastatic breast cancer, aromatase inhibitors are being tested in clinical trials to compare them to hormone therapy with tamoxifen. Examples of aromatse inhibitors currently in use include anastrozole, letrozole and exemestane.

Tyrosine kinase inhibitor therapy is a cancer treatment option that involves inhibition of kinase signalling pathways to interfere and/or halt cell growth. As discussed above, HER2 has become an important target in the search for new anti-cancer therapies and small molecule inhibitors of the kinase activity of the receptor have proven to be a valuable treatment option. The kinase inhibitors may have activity against multiple kinases. An example of this is the inhibitor lapatinib which has activity against both EGFR [ErbB1] (Kᵢ=3 nM) and HER2 [ErbB2] (Kᵢ=13 nM). Alternatively, the inhibitor may have specific activity against just HER2 [ErbB2]. An example of this is CP-724,714 with an EGFR [ErbB1] Kᵢ=6000 nM and HER2 [ErbB2] Kᵢ=7 nM. Other examples of HER2 [ErbB2] selective inhibitors include CP-654,577 and ARRY-380.

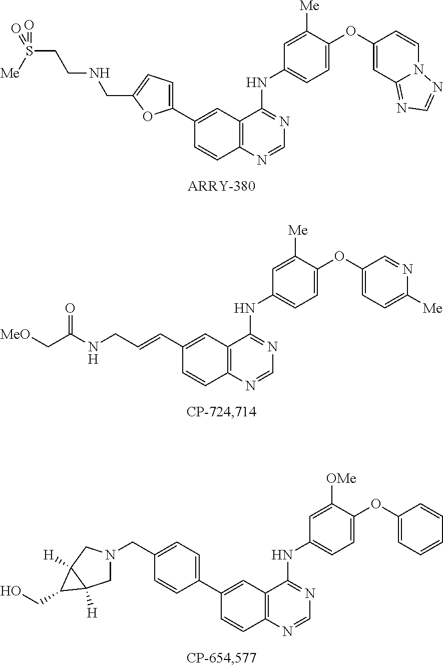

### Chemical

### Structure of HER2 (ErbB2) Selective Tyrosine Kinase Inhibitors

Monoclonal antibody therapy is a cancer treatment that uses antibodies made in the laboratory, from a single type of immune system cell. These antibodies can identify substances on cancer cells or normal substances that may help cancer cells grow. The antibodies attach to the substances and kill the cancer cells, block their growth, or keep them from spreading. Monoclonal antibodies are given by infusion. They may be used alone or to carry drugs, toxins, or radioactive material directly to cancer cells. Monoclonal antibodies are also used in combination with chemotherapy as adjuvant therapy.

### Monoclonal Antibodies to HER2

Monoclonal antibodies with affinity towards HER2 have also been a major area of research. Trastuzumab (Herceptin) is a humanized monoclonal antibody that acts on HER2. Trastuzumab is a recombinant DNA-derived humanized monoclonal antibody that selectively binds with high affinity in a cell-based assay (K_{d}=5 nM) to the extracellular domain of the human epidermal growth factor receptor 2 protein, HER2. The antibody is an IgG₁ kappa that contains human framework regions with the complementarity-determining regions of a murine antibody (4D5) that binds to HER2. The humanized antibody against HER2 is produced by a mammalian cell (Chinese Hamster Ovary [CHO]) suspension culture. A sample of the hybridoma cell line expressing this antibody (4D5) has been deposited with ATCC under the code of ATCC CRL 10463. Trastuzumab is indicated for use as part of a treatment regimen containing doxorubicin, cyclophosphamide, and paclitaxel for the adjuvant treatment of patients with HER2-overexpressing, node-positive breast cancer. Additionally, trastuzumab as a single agent is indicated for the treatment of patients with metastatic breast cancer whose tumors overexpress the HER2 protein and who have received one or more chemotherapy regimens for their metastatic disease. Also, trastuzumab in combination with paclitaxel is indicated for treatment of patients with metastatic breast cancer whose tumors overexpress the HER2 protein and who have not received chemotherapy for their metastatic disease. Trastuzumab is administered as an intravenous infusion once every 7 days. The recommended dose of trastuzumab for the first infusion is 4 mg/kg administered as a 90-minute intravenous infusion. The recommended subsequent weekly dose of 2 mg/kg can be administered as a 30-minute intravenous infusion if the first infusion was well tolerated.

### Resistance to HER2 Antibody Treatment

As discussed above, trastuzumab, a monoclonal antibody to the HER2 receptor tyrosine kinase leads to clinical responses as a single agent and improves survival when added to chemotherapy for advanced HER2-positive breast cancer. However, some patients do not respond to trastuzumab, and most eventually develop clinical resistance. Mechanisms of intrinsic and acquired trastuzumab resistance are poorly understood. One study which utilized a cell line-based approach to delineate genetic and protein alterations associated with resistance has been reported. (D. Tripathy et al Journal of Clinical Oncology, 2005 Vol 23, No 16S, 3121). These researchers studied two HER2-positive breast cancer cell lines (BT474 and SKBR3) that were serially passaged in the presence of trastuzumab until in vitro resistance was documented. Resistant cell lines emerged after 12 months and exhibited a 3-fold more rapid growth rate in the absence of trastuzumab. Following trastuzumab exposure, G₀/G₁ arrest was observed in sensitive compared to resistant cells (84 vs. 68%), with fewer cells in S-phase (3 vs. 14%). Resistant cell lines exhibited fewer changes in gene expression with trastuzumab as well as upregulation of the chemokine receptor CXCR4 and mitotic checkpoint regulators, and downregulation of PTEN compared to sensitive cells.

Thus, as provided herein, combination therapies comprising a combination of a 1,2-diphenylpyrrole derivative and a monoclonal antibody that selectively binds the HER2 receptor, may be applied with one or more other anti-tumor substances, for example, those selected from, mitotic inhibitors, for example vinblastine; alkylating agents, for example, cis-platin, carboplatin, and cyclophosphamide; anti-metabolites, for example capecitabine, 5-fluorouracil, cytosine arabinoside and hydroxyurea, or, for example, anti-metabolites such as pemetrexed, methotrexate, raltitrexed, or N-(5-[N-(3,4-dihydro-2-methyl-4-oxoquinazolin-6-ylmethyl)-N-methylamino]-2-thenyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide).

In one embodiment, the invention provides a combination of a 1,2-diphenylpyrrole derivative and a monoclonal antibody that selectively binds the HER2 receptor wherein the 1,2-diphenylpyrrole derivative is 2-(4-ethoxyphenyl)-4-methyl-1-(4-sulfamoylphenyl)-pyrrole and the monoclonal antibody that selectively binds the HER2 receptor is trastuzumab which may be applied with one or more other anti-tumor substances, for example, those selected from, mitotic inhibitors, for example vinblastine; alkylating agents, for example, cis-platin, carboplatin, and cyclophosphamide; anti-metabolites, for example capecitabine, 5-fluorouracil, cytosine arabinoside and hydroxyurea, or, for example, anti-metabolites such as pemetrexed, methotrexate, raltitrexed, or N-(5-[N-(3,4-dihydro-2-methyl-4-oxoquinazolin-6-ylmethyl)-N-methylamino]-2-thenyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide).

In the treatment of HER2/neu positive breast cancer, a combination therapy comprising a combination of 2-(4-ethoxyphenyl)-4-methyl-1-(4-sulfamoylphenyl)-pyrrole and trastuzumab can be used to treat the disease in combination with other antiangiogenic agents, or in combination with surgery, radiation therapy or with chemotherapeutic agents. Other chemotherapeutic agents include, for example, paclitaxel, doxorubicin, cyclophosphamide, lapatinib, capecitabine and CL-387785.

For the combination therapies including combination therapies having pharmaceutical compositions described herein, the effective amounts of the compound presently described herein useful for inhibiting abnormal cell growth (e.g., other antiproliferative agent, anti-angiogenic, signal transduction inhibitor or immune-system enhancer) can be determined by those of ordinary skill in the art, based on the effective amounts for the compound described herein and those known or described for the chemotherapeutic or other agent. The formulations and routes of administration for such therapies and compositions can be based on the information described herein for compositions and therapies comprising the combinations presented herein as the sole active agent and on information provided for the chemotherapeutic or other agent in combination therewith.

In one embodiment, the invention provides a method for inhibiting abnormal cell growth in a subject comprising administering to the subject an effective amount of a combination therapy comprising a combination of a 1,2-diphenylpyrrole derivative and a monoclonal antibody that selectively binds the HER2 receptor, or their pharmaceutically acceptable salt, solvate or prodrug thereof, in combination with radiation therapy effective in inhibiting abnormal cell growth in the subject. Techniques for administering radiation therapy are known to a person of skill in the art and these techniques can be used in the combination therapy described herein.

As illustrated below, the methods and therapies disclosed herein have shown superior results compared to a combination based on other COX-2 inhibitors. For example combinations disclosed herein, based on a combination of 2-(4-ethoxyphenyl)-4-methyl-1-(4-sulfamoylphenyl)-pyrrole and trastuzumab, have shown 100% increase in tumor growth delay compared to a combination including celecoxib and trastuzumab. Combinations containing 2-(4-ethoxyphenyl)-4-methyl-1-(4-sulfamoylphenyl)-pyrrole at a dose from about 5 to about 25 mg/kg and trastuzumab have shown significant synergism effects. For example, combinations containing 2-(4-ethoxyphenyl)-4-methyl-1-(4-sulfamoylphenyl)-pyrrole at a dose of about 10 mg/kg and trastuzumab at a dose of 15 mg/kg increased tumor growth delay by 100% compared to administration of trastuzumab alone. On the other hand, a combination containing celecoxib and tratusumab showed no significant effect on tumor growth delay when compared to administration of trastuzumab alone.

### 2-(4-Ethoxyphenyl)-4-methyl-1-(4-sulfamoylphenyl)-pyrrole

2-(4-Ethoxyphenyl)-4-methyl-1-(4-sulfamoylphenyl)-pyrrole is a COX-2 selective inhibitor. U.S. Pat. No. 6,887,893 and RE39,420 describe the preparation of 2-(4-ethoxyphenyl)-4-methyl-1-(4-sulfamoylphenyl)-pyrrole and other chemically-related compounds.

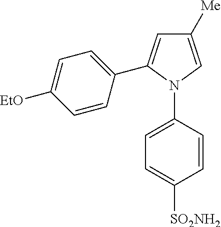

### Chemical Structure of 2-(4-ethoxyphenyl)-4-methyl-1-(4-sulfamoylphenyl)-pyrrole

The compositions provided herein may be enantiomerically pure, such as a single enantiomer or a single diastereomer, or be stereoisomeric mixtures, such as a mixture of enantiomers, a racemic mixture, or a diastereomeric mixture, or a polymorph of the active agent. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the composition described herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The composition described herein may also be provided as a prodrug, which is a functional derivative of the 1,2-diphenylpyrrole derivative and/or the inhibitor of HER2 [ErbB2] and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The combinations presently described herein may also be useful in the treatment of additional disorders in which aberrant expression ligand/receptor interactions or activation or signalling events related to various protein tyrosine kinases are involved. Such disorders may include those of neuronal, glial, astrocytal, hypothalamic, glandular, macrophagal, epithelial, stromal, or blastocoelic nature in which aberrant function, expression, activation or signalling of the erbB tyrosine kinases are involved. In addition, the combinations presented herein may have therapeutic utility in inflammatory, angiogenic and immunologic disorders involving both identified and as yet unidentified tyrosine kinases that are inhibited by the combinations presented herein.

### Pharmaceutical Compositions

Provided herein are pharmaceutical compositions comprising a combination of a 1,2-diphenylpyrrole derivative and an inhibitor of HER2 [ErbB2] as an active ingredient, or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers.

Also provided herein are pharmaceutical compositions comprising a combination of a 1,2-diphenylpyrrole derivative and an inhibitor of HER2 [ErbB2] as an active ingredient, or a pharmaceutically acceptable salt, solvate, or prodrug thereof, in a pharmaceutically acceptable vehicle, carrier, diluent, or excipient, or a mixture thereof; and one or more release controlling excipients as described herein. Provided herein are pharmaceutical compositions comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577 as an active ingredient, or a pharmaceutically acceptable salt, solvate, or prodrug thereof, in a pharmaceutically acceptable vehicle, carrier, diluent, or excipient, or a mixture thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. The pharmaceutical compositions may also comprise non-release controlling excipients.

Provided herein are pharmaceutical compositions in film-coated dosage forms, which comprise a combination of a 1,2-diphenylpyrrole derivative and an inhibitor of HER2 [ErbB2] as an active ingredient, or a pharmaceutically acceptable salt, solvate, or prodrug thereof; and one or more tabletting excipients to form a tablet core using conventional tabletting processes and subsequently coating the core. The tablet cores can be produced using conventional granulation methods, for example wet or dry granulation, with optional comminution of the granules and with subsequent compression and coating. Granulation methods are described, for example, in Voigt, pages 156-69.

Suitable excipients for the production of granules are, for example pulverulent fillers optionally having flow-conditioning properties, for example talcum, silicon dioxide, for example synthetic amorphous anhydrous silicic acid of the Syloid® type (Grace), for example SYLOID 244 FP, microcrystalline cellulose, for example of the Avicel® type (FMC Corp.), for example of the types AVICEL PH101, 102, 105, RC581 or RC 591, Emcocel® type (Mendell Corp.) or Elcema® type (Degussa); carbohydrates, such as sugars, sugar alcohols, starches or starch derivatives, for example lactose, dextrose, saccharose, glucose, sorbitol, mannitol, xylitol, potato starch, maize starch, rice starch, wheat starch or amylopectin, tricalcium phosphate, calcium hydrogen phosphate or magnesium trisilicate; binders, such as gelatin, tragacanth, agar, alginic acid, cellulose ethers, for example methylcellulose, carboxymethylcellulose or hydroxypropylmethylcellulose, polyethylene glycols or ethylene oxide homopolymers, especially having a degree of polymerization of approximately from 2.0×10³ to 1.0×10⁵ and an approximate molecular weight of about from 1.0×10⁵ to 5.0×10⁶, for example excipients known by the name Polyox® (Union Carbide), polyvinylpyrrolidone or povidones, especially having a mean molecular weight of approximately 1000 and a degree of polymerization of approximately from about 500 to about 2500, and also agar or gelatin; surface-active substances, for example anionic surfactants of the alkyl sulfate type, for example sodium, potassium or magnesium n-dodecyl sulfate, n-tetradecyl sulfate, n-hexadecyl sulfate or n-octadecyl sulfate, of the alkyl ether sulfate type, for example sodium, potassium or magnesium n-dodecyloxyethyl sulfate, n-tetradecyloxyethyl sulfate, n-hexadecyloxyethyl sulfate or n-octadecyloxyethyl sulfate, or of the alkanesulfonate type, for example sodium, potassium or magnesium n-dodecanesulfonate, n-tetradecanesulfonate, n-hexadecanesulfonate or n-octadecanesulfonate, or non-ionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the Pluronics® (BWC) or Synperonic® (ICI) type

Further provided herein are pharmaceutical compositions in enteric coated dosage forms, which comprise a combination of a 1,2-diphenylpyrrole derivative and an inhibitor of HER2 [ErbB2] as an active ingredient, or a pharmaceutically acceptable salt, solvate, or prodrug thereof; and one or more release controlling excipients for use in an enteric coated dosage form. Provided herein are pharmaceutical compositions in enteric coated dosage forms comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, as an active ingredient, or a pharmaceutically acceptable salt, solvate, or prodrug thereof, in a pharmaceutically acceptable vehicle, carrier, diluent, or excipient, or a mixture thereof; and one or more release controlling excipients for use in an enteric coated dosage form. The pharmaceutical compositions may also comprise non-release controlling excipients.

Further provided herein are pharmaceutical compositions in effervescent dosage forms, which comprise a combination of a 1,2-diphenylpyrrole derivative and an inhibitor of HER2 [ErbB2] as an active ingredient, or a pharmaceutically acceptable salt, solvate, or prodrug thereof; and one or more release controlling excipients for use in effervescent dosage forms. Also provided herein are pharmaceutical compositions in effervescent dosage forms comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577 as an active ingredient, or a pharmaceutically acceptable salt, solvate, or prodrug thereof, in a pharmaceutically acceptable vehicle, carrier, diluent, or excipient, or a mixture thereof; and one or more release controlling excipients for use in an effervescent dosage forms. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 hour up to 24 hours. The pharmaceutical compositions comprise a combination of a 1,2-diphenylpyrrole derivative and an inhibitor of HER2 [ErbB2] as an active ingredient, or a pharmaceutically acceptable salt, solvate, or prodrug thereof; and one or more release controlling and non-release controlling excipients, such as those excipients suitable for a disruptable semi-permeable membrane and as swellable substances. Additionally, the invention provides pharmaceutical compositions comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577 as an active ingredient, or a pharmaceutically acceptable salt, solvate, or prodrug thereof; and one or more release controlling and non-release controlling excipients, such as those excipients suitable for a disruptable semi-permeable membrane and as swellable substances.

Provided herein also are pharmaceutical compositions in a dosage form for oral administration to a subject, which comprises a combination of a 1,2-diphenylpyrrole derivative and an inhibitor of HER2 [ErbB2] as an active ingredient, or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer. Additionally, the invention provides pharmaceutical compositions in a dosage form for oral administration to a subject comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577 enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise a combination of a 1,2-diphenylpyrrole derivative and an inhibitor of HER2 [ErbB2] as an active ingredient, or a pharmaceutically acceptable salt, solvate, or prodrug thereof, in the form of enteric-coated granules, as delayed-release capsules for oral administration. In one embodiment the invention provides for pharmaceutical compositions that comprise a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is CP-724714 and wherein the quantity of 2-(4-ethoxyphenyl)-4-methyl-1-(4-sulfamoylphenyl)-pyrrole present in the composition is from about 100 mg to about 1200 mg and the quantity of CP-724714 present in the composition is from about 50 mg to about 500 mg wherein both 2-(4-ethoxyphenyl)-4-methyl-1-(4-sulfamoylphenyl)-pyrrole and CP-724714 are present as an active ingredient, or a pharmaceutically acceptable salt, solvate, or prodrug thereof, in the form of enteric-coated granules, as delayed-release capsules for oral administration. In additional embodiments, the composition may contain about 100 mg, about 200 mg, about 300 mg, about 400 mg, about 500 mg, about 600 mg, about 700 mg, about 800 mg, about 900 mg, about 1000 mg, about 1100 mg or about 1200 mg of 2-(4-ethoxyphenyl)-4-methyl-1-(4-sulfamoylphenyl)-pyrrole as an active ingredient, or a pharmaceutically acceptable salt, solvate, or prodrug thereof. In additional embodiments, the composition may contain about 50 mg, about 125 mg, about 250 mg, about 375 mg or about 500 mg of CP-724714 as an active ingredient, or a pharmaceutically acceptable salt, solvate, or prodrug thereof.

The pharmaceutical compositions may further comprise glyceryl monostearate 40-50, hydroxypropyl cellulose, hypromellose, magnesium stearate, methacrylic acid copolymer, sugar spheres, talc, carnauba wax, crospovidone, diacetylated monoglycerides, ethylcellulose, hypromellose phthalate, mannitol, sodium hydroxide, sodium stearyl fumarate, titanium dioxide, yellow ferric oxide, calcium stearate, hydroxypropyl methylcellulose, iron oxide, polysorbate 80, povidone, propylene glycol, sodium carbonate, sodium lauryl sulfate, and triethyl citrate.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compositions provided herein may be administered alone, or in combination with one or more other compounds provided herein, one or more other active ingredients. The pharmaceutical compositions that comprise a compound provided herein may be formulated in various dosage forms for oral, parenteral, buccal, intranasal, epidural, sublingual, pulmonary, local, rectal, transdermal, or topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the combinations may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the combinations may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

As described herein, the compositions and methods for using the composition comprising a combination of a 1,2-diphenylpyrrole derivative and an inhibitor of HER2 [ErbB2], may be formulated without carriers or excipients or may be combined with one or more pharmaceutically acceptable carriers for administration. For example, solvents, diluents and the like, and may be administered orally in such forms as tablets, capsules, dispersible powders, granules, or suspensions containing, for example, from about 0.05 to about 5% of suspending agent, syrups containing, for example, from about 10 to about 50% of sugar, and elixirs containing, for example, from about 20 to about 50% ethanol, and the like. Such pharmaceutical preparations may contain, for example, from about 0.05 up to about 90% of the active ingredient in combination with the carrier, more usually between about 5% and about 60% by weight. Also, the compositions and methods for using the composition comprising a combination of a 1,2-diphenylpyrrole derivative and an inhibitor of HER2 [ErbB2] wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577, may be formulated without carriers or excipients or may be combined with one or more pharmaceutically acceptable carriers for administration.

The effective dosage of each active ingredient employed may vary depending on the particular compound employed, the mode of administration and the severity of the condition being treated. The projected daily dosage of the inhibitor of HER2 [ErbB2] will depend on its potency. Similarly, the dosage of the 1,2-diphenylpyrrole derivative inhibitor used depends on the relative potency of 1,2-diphenylpyrrole derivative inhibitor, compared for example to sulindac. Numerous methods for evaluating and comparing 1,2-diphenylpyrrole derivative inhibitor potency are known to one of skill in the art. In one embodiment, an oral daily dosage of the 1,2-diphenylpyrrole derivative inhibitor is in the range of about 100 to about 1200 mg, and the projected daily dosage of the inhibitor of HER2 [ErbB2] is in the range of about 100 to about 1000 mg. In another embodiment, an oral daily dosage of 2-(4-ethoxyphenyl)-4-methyl-1-(4-sulfamoylphenyl)-pyrrole is in the range of about 100 to about 1200 mg and the projected daily dosage of CP-724714 is in the range of about 100 to about 1000 mg. This dosage regimen may be adjusted to provide the optimal therapeutic response. For example, several divided doses may be administered daily or the dose may be proportionally reduced as indicated by the exigencies of the therapeutic situation. The 1,2-diphenylpyrrole derivative inhibitor and the inhibitor of HER2 [ErbB2] may also be administered as a combined dosage unit, or as separate components. When administered as separate components, each component may be administered at the same time, or at different times during the treatment period.

It is understood, however, that a specific dose level for any particular patient will depend upon a variety of factors such as, for example, decreases in the liver and kidney function.

Treatment dosages generally may be titrated to optimize safety and efficacy. Typically, dosage-effect relationships from in vitro studies initially can provide useful guidance on the proper doses for patient administration. Studies in animal models also generally may be used for guidance regarding effective dosages for treatment of cancers in accordance with the present disclosure. In terms of treatment protocols, it should be appreciated that the dosage to be administered will depend on several factors, including the particular agent that is administered, the route administered, the condition of the particular patient, etc. Determination of these parameters are well within the skill of the art. These considerations, as well as effective formulations and administration procedures are well known in the art and are described in standard textbooks.

### Oral Formulations

Oral formulations containing the active combinations described herein may comprise any conventionally used oral forms, including: tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

### Oral Administration

As described herein, the combination regimen can be given simultaneously or can be given in a staggered regimen, with a 1,2-diphenylpyrrole derivative being given at a different time during the course of chemotherapy than an inhibitor of HER2 [ErbB2]. This time differential may range from several minutes, hours, days, weeks, or longer between administration of the two compounds. Therefore, the term combination does not necessarily mean administered at the same time or as a unitary dose, but that each of the components are administered during a desired treatment period. The agents may also be administered by different routes. As is typical for chemotherapeutic regimens, a course of chemotherapy may be repeated several weeks later, and may follow the same timeframe for administration of the two compounds, or may be modified based on patient response.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

In other embodiments, the pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

### Parenteral Administration

In some embodiments, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In other embodiments, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In some embodiments, the pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### Modified Release

In other embodiments, the pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

In some embodiments, the pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

In other embodiments, the pharmaceutical compositions provided herein in a modified release dosage form may be prepared by methods known to those skilled in the art, including direct compression, dry or wet granulation followed by compression, melt-granulation followed by compression.

### 2. Osmotic Controlled Release Devices

In some embodiments, the pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredients) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semi-permeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semi-permeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semi-permeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semi-permeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In other embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

In some embodiments, the pharmaceutical compositions provided herein in a modified release dosage form may be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

In some embodiments, the pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874, all of which are incorporated herein by their entirety.

### Immediate Release

In some embodiments, the pharmaceutical compositions provided herein in an immediate release dosage form are capable of releasing not less than 75% of the therapeutically active ingredient or combination and/or meet the disintegration or dissolution requirements for immediate release tablets of the particular therapeutic agents or combination included in the tablet core, as set forth in USP XXII, 1990 (The United States Pharmacopeia.)

### Topical Administration

In other embodiments, the pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

In further embodiments, the pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

In some embodiments, the pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or an surfactant, such as sorbitan trioleate, oleic acid, or an oligolactic acid.

In another embodiment, the pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

In one embodiment, the pharmaceutical compositions provided herein for topical administration may be formulated to be immediate release or modified release, including delayed-, sustained-, pulsed-, controlled-, targeted, and programmed release.

### EXAMPLES

### Example 1

### Synthesis of 2-(4-ethoxyphenyl)-4-methyl-1-(4-sulfamoylphenyl)-pyrrole

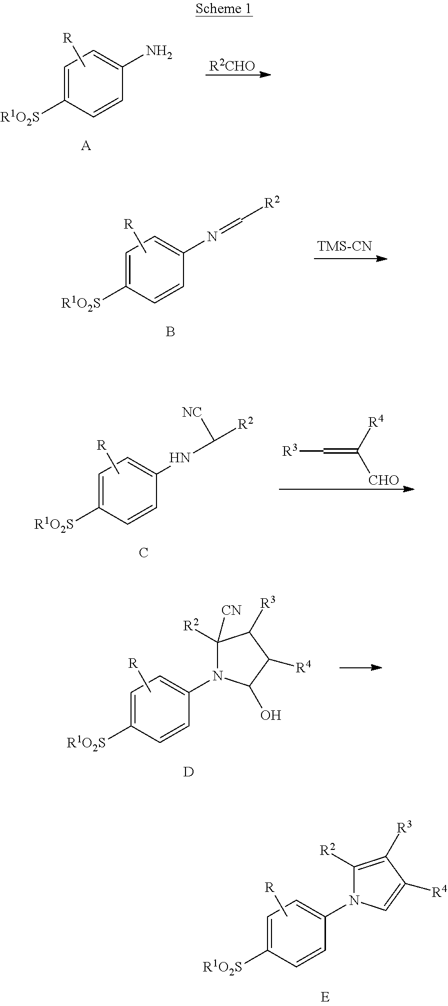

Substituted benzaldehyde undergoes dehydration condensation by reaction with aniline compound A in an inert solvent at a temperature of between 5° C. to 200° C. to give aldimine compound B. Trimethylsilyl cyanide is then reacted with aldimine compound B in the presence of a Lewis acid to afford anilinonitrile C. An α,β-unsaturated aldehyde is then reacted with anilinonitrile C to afford compound D which then undergoes dehydration and dehydrogencyanation under basic conditions in a modification of the method described in Ann. Chem. 589, 176 (1954).

### Example 2

### Synthesis of CP-724714

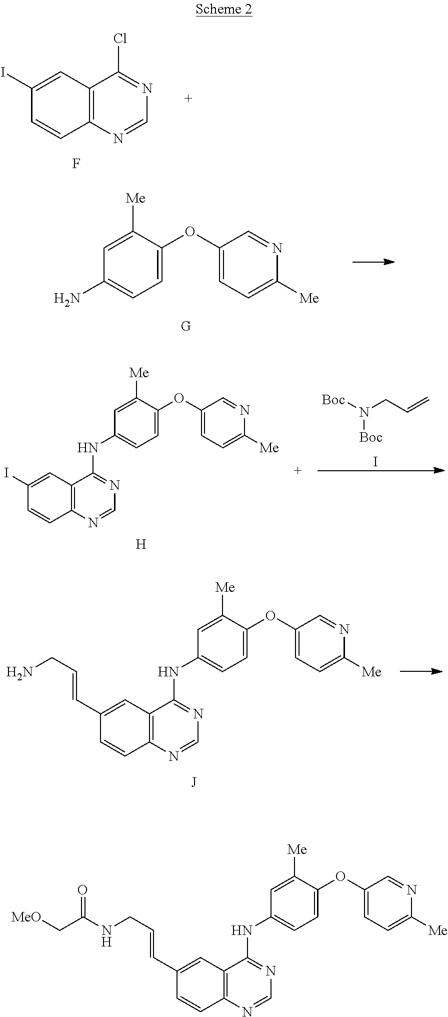

According to the methods of Ripin et al (Org. Process Res. Dev. 2005, 9, 440), starting compound F is condensed with aniline G under basic conditions such as K₂CO₃ in DMF as solvent. Iodoquinazoline H is subjected to a palladium (0) catalyzed coupling reaction with protected allylamine I followed by acid promoted deprotection to afford heterocycle J. Acetylation of J with methoxyacetyl chloride will give CP-724,714.

### Example 3

### Production of Anti-HER2 Monoclonal Antibodies

Five female Balb/c mice were immunized with HER2 amplified NIH 3T3 transformed cells over a period of 22 weeks. The first four injections each had approximately 10⁷ cells/mouse. They were administered intraperitoneally in half a milliliter of PBS on weeks 0, 2, 5, 7. Injections five and six were with a wheat germ agglutinin partially purified membrane preparation which had a whole protein concentration of about 700 μg/ml.

A 100 μl/injection was administered to each mouse intraperitoneally on weeks 9 and 13. The last injection was also with the purified material but was administered three days prior to the date of fusion intravenously.

Bleeds from the mice were tested at various times in a radioimmunoprecipitation using whole cell lysates. The three mice with the highest antibody titers were sacrificed and spleens were fused with the mouse myeloma cell line X63-Ag8.653 using the general procedure of Mishell & Shiigi, Selected Methods in Cellular Immunology, W.H. Freeman & Co., San Francisco, p. 357-363 (1980) with the following exceptions. Cells were plated at a density of approximately 2×10⁵ cells/well into ten 96 well microtiter plates. Hybrids were selected using hypoxanthine-azoserine rather than hypoxanthine-aminoptern-thymidine (HAT).

Hybridoma supernatants were tested for presence of antibodies specific for HER2 receptor by ELISA and radioimmunoprecipitation.

For the ELISA, 3.5 μg/ml of the HER2 receptor (purified on the wheat germ agglutinin column) in PBS was adsorbed to immulon II microtiter plates overnight at 4.degree. C. or for 2 hours at room temperature. Plates were then washed with phosphate buffered saline with 0.05% Tween 20 (PBS-TW20) to remove unbound antigen. Remaining binding sites were then blocked with 200 μl per well of 1% bovine serum albumin (BSA) in PBS-TW20 and incubated 1 hour at room temperature. Plates were washed as above and 100 μl of hybridoma supernatant was added to each well and incubated for 1 hour at room temperature.

Plates were washed again and 100 μl per well of an appropriate dilution of goat anti-mouse immunoglobulin coupled to horseradish peroxidase was added. The plates were incubated again for 1 hour at room temperature and then washed as above. O-phenylene diamine was added as substrate, incubated for 15-20 minutes at room temperature and then the reaction was stopped with 2.5 M H₂SO₄. The absorbance of each well was then read at 492 nm.

For the radioimmunoprecipitation, first the wheat germ purified HER2 receptor preparation was autophosphorylated in the following manner: a kinase solution with the following final concentrations was made: 0.18 mCi/ml .gamma.P³²-ATP (Amersham), 0.4 mM MgCl₂ 0.2 mM MnCl₂, 10 μM ATP, 35 μg/ml total protein concentration of partially purified HER2 all diluted in 20 mM Hepes, 0.1% triton 10% glycerol buffer (HTG). This reaction was incubated for 30 minutes at room temperature. 50 μl hybridoma supernatant was then added to 50 μl of the kinase reaction and incubated 1 hour at room temperature. 50 μl of goat anti-mouse IgG precoated protein-A sepharose CM4B, at a sepharose concentration of 80 mg/ml, was added to each sample and incubated 1 hour at room temperature.

The resulting immunocomplexes were then washed by centrifugation twice with HTG buffer and finally with 0.2% deoxycholate 0.2% Tween 20 in PBS, in a microfuge and aspirated between washes. Reducing sample buffer was added to each sample and samples were heated at 95.degree. C. for 2-5 minutes, insoluble material was removed by centrifugation and the reduced immunocomplex was loaded onto a 7.5% polyacrylamide gel containing SDS. The gel was run at 30 amp constant current and an autoradiograph was obtained from the finished gel.

Approximately 5% of the total well supernatants reacted with the HER2 receptor in the ELISA and/or radioimmunoprecipitation. From this initial 5% (about 100), some hybrids produced low affinity anti bodies and others suffered from instability and stopped secreting antibodies leaving a total of 10 high affinity stable HER2 specific antibody producing cell lines. These were expanded and cloned by limiting dilution (Oi, V. T. and Herzenberg, L. A., “Immunoglobulin Producing Hybrid Cell Lines” in Selected Methods in Cellular Immunology, p. 351-372 Mishell, B. B. and Shiigi, S. M. (eds.), W.H. Freeman and Co. (1980)). Large quantities of specific monoclonal antibodies were produced by injection of cloned hybridoma cells in pristaned primed mice to produce ascitic tumors. Ascites were then collected and purified over a protein-A sepharose column.

### Example 4

### Pharmacokinetics and Metabolism of 2-(4-ethoxyphenyl)-4-methyl-1-(4-sulfamoylphenyl)-pyrrole

Orally administered 2-(4-ethoxyphenyl)-4-methyl-1-(4-sulfamoylphenyl)-pyrrole is rapidly absorbed in all species examined (mice, rats, dogs, and monkeys). Peak plasma concentrations were achieved between 1 and 3 hours after a dose of 5 mg/kg. The elimination half life (t_{1/2}) was 4-5 hours in rodents and dogs, and approximately 2 hours in monkeys. Oral availability was greatest in rodent, and was reduced in dogs and monkeys (59 and 34% respectively). Pharmacokinetics in human subjects demonstrated a linear dose exposure relationship from doses of 2 mg to 800 mg given orally. The half-life in human subjects is 15-18 hours.

### Example 5

### Toxicology of 2-(4-ethoxyphenyl)-4-methyl-1-(4-sulfamoylphenyl)-pyrrole

Toxicological evaluation of 2-(4-ethoxyphenyl)-4-methyl-1-(4-sulfamoylphenyl)-pyrrole in mice, rats, dogs and monkeys revealed expected findings related to inhibition of cyclooxygenase and consistent with animal safety observations with other COX-2 selective inhibitors. In single dose studies, the minimum lethal dose of 2-(4-ethoxyphenyl)-4-methyl-1-(4-sulfamoylphenyl)-pyrrole was 600 mg/kg in rats and >2000 mg/kg in dogs. An endoscopy study conducted in human subjects demonstrated no increase in gastric or duodenal toxicity compared to placebo.

### Example 6

In Vitro Inhibition of HER2 [ErbB2] Kinase Activity

The in vitro activity of the combinations described herein in inhibiting the HER2 [ErbB2] receptor tyrosine kinase may be determined by the following procedure. The HER2 [ErbB2] recombinant intracellular domain (amino acids 675-1255) is expressed in baculovirus-infected Sf9 cells as a glutathione S-transferase fusion protein. The protein is purified by affinity chromatography on glutathione Sepharose beads for use in the assay. Nunc MaxiSorp 96-well plates were coated by incubation overnight at 37° C. with 100 μl/well of 0.25 mg/ml poly(Glu:Tyr, 4:1), (PGT; Sigma Chemical Co.) in PBS. Excess PGT is removed by aspiration and the plate is washed 3 times with wash buffer (0.1% Tween 20 in PBS). The kinase reaction is performed in 50 μl of 50 mM HEPES (pH 7.4) containing 125 mM sodium chloride, 10 mM magnesium chloride, 0.1 mM sodium orthovanadate, 1 mM ATP, and about 15 ng of recombinant protein. The test composition in DMSO is added; the final DMSO concentration is 2.5%. Phosphorylation is initiated by addition of ATP and allowed to proceeded for 6 min at room temperature, with constant shaking. The kinase reaction is terminated by aspiration of the reaction mixture and washing four times with wash buffer. Phosphorylated PGT is measured after a 25-min incubation with 50 μl/well HRP conjugated-PY54 (Oncogene Science Inc. Pharmaceuticals, Uniondale, N.Y.) antiphosphotyrosine antibody, diluted to 0.2 μg/ml in blocking buffer (3% BSA, 0.05% Tween 20 in PBS). Antibody is removed by aspiration and the plate is washed four times with wash buffer. The colorimetric signal is developed by addition of 50 μl/well Tetramethylbenzidine Microwell Peroxidase Substrate (Kirkegaard and Perry Labs, Gaithersburg, Md.) and stopped by the addition of 50 μl/well 0.09 M sulfuric acid. The phosphotyrosine product formed is estimated by measurement of absorbance at 450 nm. The signal for controls is typically A0.6-1.2, with essentially no background in wells without ATP, kinase protein, or PGT, and is proportional to the time of incubation for 6 min.

### Example 7

### Pharmaceutical Compositions and Dosage Forms

Dosage formulations comprising pharmaceutical excipients and carriers and a pharmaceutical composition comprising a combination of CP-724714 (A) and 2-(4-ethoxyphenyl)-4-methyl-1-(4-sulfamoylphenyl)-pyrrole (B) include:

| | Amount of A per tablet | Amount of B per tablet |
|---|---|---|
| Combination | (mg) | (mg) |
| A/B | 50 | 100, 200, 300, 400, 500, 600, |
| | | 700, 800, 900, 1000, 1100, |
| | | 1200 |
| A/B | 125 | 100, 200, 300, 400, 500, 600, |
| | | 700, 800, 900, 1000, 1100, |
| | | 1200 |
| A/B | 250 | 100, 200, 300, 400, 500, 600, |
| | | 700, 800, 900, 1000, 1100, |
| | | 1200 |
| A/B | 375 | 100, 200, 300, 400, 500, 600, |
| | | 700, 800, 900, 1000, 1100, |
| | | 1200 |
| A/B | 500 | 100, 200, 300, 400, 500, 600, |
| | | 700, 800, 900, 1000, 1100, |
| | | 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and CP-724714 or as a separate administration of a single dose of 2-(4-ethoxyphenyl)-4-methyl-1-(4-sulfamoylphenyl)-pyrrole and a single dose of CP-724714.

### Example 8

### Biological Evaluation

### SK-BR-3 Model:

Mice are injected subcutaneously in the left paw (1×10⁶ tumor cells suspended in 30% Matrigel) and tumor volume is evaluated using a phlethysmometer twice a week for 30-60 days. Implantation of human breast cancer cells (SK-BR-3) into nude mice produces tumors that will reach 0.6-2 ml between 30-50 days. Blood is drawn twice during the experiment in a 24 h protocol to assess plasma concentration and total exposure by AUC analysis. The data is expressed as the mean+/−SEM. Student's and Mann-Whitney tests are used to assess differences between means using the InStat software package.

A. Mice injected with SK-BR-3 cancer cells are treated with cytoxin i.p at doses of 50 mg/kg on days 5, 7 and 9 in the presence or absence of a combination therapy comprising a combination of 2-(4-ethoxyphenyl)-4-methyl-1-(4-sulfamoylphenyl)-pyrrole with in the diet and trastuzumab given intravenous. The efficacy of both agents are determined by measuring tumor volume. The results from these studies may demonstrate that administration of a combination therapy comprising a combination of 2-(4-ethoxyphenyl)-4-methyl-1-(4-sulfamoylphenyl)-pyrrole with trastuzumab to tumor bearing mice can delay the growth of tumors and metastasis.

B. In a second assay, mice injected with SK-BR-3 cancer cells are then treated with 5-FU on days 12 through 15. Mice injected with SK-BR-3 cancer cells are treated with 5-FU i.p at doses of 50 mg/kg on days 12, 13, 14, and 15 in the presence or absence of a combination therapy comprising a combination of 2-(4-ethoxyphenyl)-4-methyl-1-(4-sulfamoylphenyl)-pyrrole in the diet and trastuzumab administered intravenously. The efficacy of both agents are determined by measuring tumor volume. Treatment using the combination therapy may reduce tumor volume by up to 70%. In the same assay, 5-FU decreases tumor volume by 61%. Further, the composition and 5-FU may decrease tumor volume by 83%.

C. In a third assay, mice injected with SK-BR-3 breast cancer cells are treated with 5-FU i.p 50 mg/kg on days 14 through 17 in the presence or absence of a composition comprising a combination of 2-(4-ethoxyphenyl)-4-methyl-1-(4-sulfamoylphenyl)-pyrrole and valdecoxib in the diet and trastuzumab administered intravenously. The efficacy of both agents are determined by measuring tumor volume. Treatment with 5-FU may result in a 35% reduction in tumor volume. Treatment with the composition and valdecoxib may reduce tumor volume by 52% and 69%, respectively. In the same assay, the combination of 5-FU and the composition may decrease tumor volume by 72% while the combination of 5-FU and valdecoxib may decrease tumor volume by 74%.

### Example 9

### Combinations based on 2-(4-ethoxyphenyl)-4-methyl-1-(4-sulfamoylphenyl)-pyrrole and trastuzumab provide increased tumor growth delay

### BT474 Model:

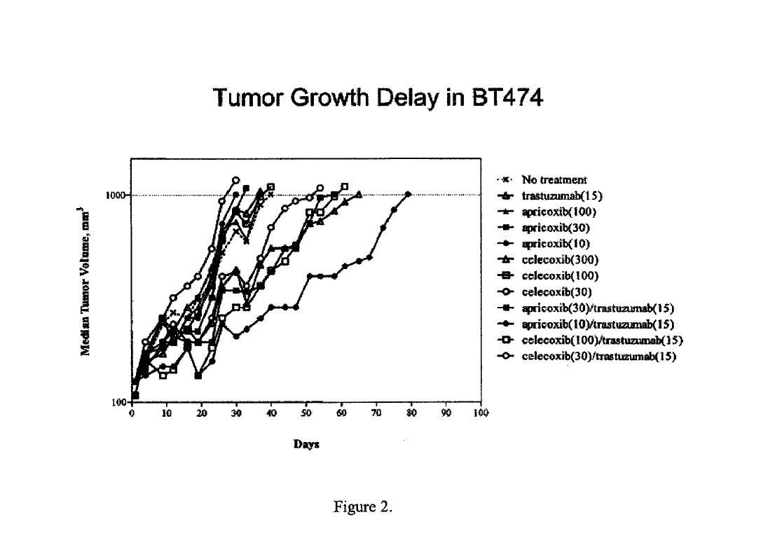

Female SCID mice were injected subcutaneously in the flank (1 mm³ BT474 tumor) and tumor volume was evaluated using a caliper twice a week for up to 90 days, longer for responders. Upon reaching an average tumor size of 100-200 mg the treatment was begun. Celecoxib and 2-(4-ethoxyphenyl)-4-methyl-1-(4-sulfamoylphenyl)-pyrrole were both formulated as a solution in 1% carboxymethyl cellulose/water and dosed orally by gavage. Trastuzumab was dosed in 100% saline. The endpoint for the study was when tumor volume reached 0.75 gms or 90 days, which ever came first. Mice injected with BT474 cancer cells were grouped into one of the following treatment groups:
Group 0: no treatment
Group 1: trastuzumab (15 mg/kg, ip, biwk×3);
Group 2: 2-(4-ethoxyphenyl)-4-methyl-1-(4-sulfamoylphenyl)-pyrrole (100 mg/kg, po, qd to end);
Group 3: 2-(4-ethoxyphenyl)-4-methyl-1-(4-sulfamoylphenyl)-pyrrole (30 mg/kg, po, qd to end);
Group 4: 2-(4-ethoxyphenyl)-4-methyl-1-(4-sulfamoylphenyl)-pyrrole (10 mg/kg, po, qd to end);
Group 5: celecoxib (300 mg/kg, po, qd to end);
Group 6: celecoxib (100 mg/kg, po, qd to end);
Group 7: celecoxib (30 mg/kg, po, qd to end);
Group 8: 2-(4-ethoxyphenyl)-4-methyl-1-(4-sulfamoylphenyl)-pyrrole (30 mg/kg, po, qd to end) and trastuzumab (15 mg/kg, ip, biwk×3);
Group 9: 2-(4-ethoxyphenyl)-4-methyl-1-(4-sulfamoylphenyl)-pyrrole (10 mg/kg, po, qd to end) and trastuzumab (15 mg/kg, ip, biwk×3);
Group 10: celecoxib (100 mg/kg, po, qd to end) and trastuzumab (15 mg/kg, ip, biwk×3);
Group 11: celecoxib (30 mg/kg, po, qd to end) and trastuzumab (15 mg/kg, ip, biwk×3).
   Response summary for BT474 study (see also FIG. 2)

| | | Median | | | | |
|---|---|---|---|---|---|---|
| | | Total | Differ- | % | | |
| | | Time to | ence | tumor | | Regressions |
| | | Endpoint | from | growth | Statistical | (PR/CR/ |
| Group | n | (days) | control | delay | Significance | TFS) |
| 0 | 6 | 38.2 | — | — | — | 0/0/0 |
| 1 | 10 | 56.8 | 18.6 | 49 | * | 0/1/0 |
| 2 | 10 | 32.7 | −5.5 | −14 | ne | 0/0/0 |
| 3 | 7 | 32.0 | −6.2 | −16 | ns | 0/0/0 |
| 4 | 7 | 29.7 | −8.5 | −22 | ns | 0/0/0 |
| 5 | 8 | 36.6 | −1.6 | −4 | ns | 0/0/0 |
| 6 | 6 | 36.8 | −1.4 | −4 | ns | 0/0/0 |
| 7 | 7 | 27.5 | −10.7 | −28 | ** | 0/0/0 |
| 8 | 10 | 54.4 | 16.2 | 42 | ** | 0/0/0 |
| 9 | 10 | 78.1 | 39.9 | 104 | *** | 1/2/2 |
| 10 | 10 | 55.4 | 17.2 | 45 | ns | 0/0/0 |
| 11 | 10 | 50.0 | 11.8 | 31 | ns | 0/0/0 |

ne: not evaluable;
ns: P > 0.05;
*: 0.01 < P < 0.05;
**: 0.001 < P < 0.01;
***: P < 0.001

Compared to trastuzumab alone, a 2-fold increase in tumor growth delay was observed with the combination of 2-(4-ethoxyphenyl)-4-methyl-1-(4-sulfamoylphenyl)-pyrrole and trastuzumab (group 9). This combination of 2-(4-ethoxyphenyl)-4-methyl-1-(4-sulfamoylphenyl)-pyrrole and trastuzumab had 1 partial regressions, 2 complete regression and 2 tumor free survivor, while trastuzumab alone had 0 partial regressions, 1 complete regression and 1 tumor free survivor. Celecoxib demonstrated no added benefit upon combination with trastuzumab.

### Example 10

### Dose Response of 2-(4-ethoxyphenyl)-4-methyl-1-(4-sulfamoylphenyl)-pyrrole and Trastuzumab Combinations

### BT474 Model:

Female SCID mice were injected subcutaneously in the flank (1 mm³ BT474 tumor) and tumor volume was evaluated using a caliper twice a week for up to 90 days, longer for responders. Upon reaching an average tumor size of 100-200 mg the treatment was begun. 2-(4-Ethoxyphenyl)-4-methyl-1-(4-sulfamoylphenyl)-pyrrole was formulated as a solution in 1% carboxymethyl cellulose/water and dosed orally by gavage. Trastuzumab was dosed in 100% saline. The endpoint for the study was when tumor volume reached 0.75 gms or 90 days, which ever came first. Mice injected with BT474 cancer cells were grouped into one of the following treatment groups:
Group 1: no treatment
Group 2: trastuzumab (15 mg/kg, ip, biwk×3);
Group 3: 2-(4-ethoxyphenyl)-4-methyl-1-(4-sulfamoylphenyl)-pyrrole (40 mg/kg, po, qd to end) and trastuzumab (15 mg/kg, ip, biwk×3);
Group 4: 2-(4-ethoxyphenyl)-4-methyl-1-(4-sulfamoylphenyl)-pyrrole (20 mg/kg, po, qd to end) and trastuzumab (15 mg/kg, ip, biwk×3);
Group 5: 2-(4-ethoxyphenyl)-4-methyl-1-(4-sulfamoylphenyl)-pyrrole (10 mg/kg, po, qd to end) and trastuzumab (15 mg/kg, ip, biwk×3);
Group 6: 2-(4-ethoxyphenyl)-4-methyl-1-(4-sulfamoylphenyl)-pyrrole (5 mg/kg, po, qd to end) and trastuzumab (15 mg/kg, ip, biwk×3).
   Response summary for BT474 dose response study

| | | Median | | | | |
|---|---|---|---|---|---|---|
| | | Total | Differ- | % | | |
| | | Time to | ence | tumor | | Regressions |
| | | Endpoint | from | growth | Statistical | (PR/CR/ |
| Group | n | (days) | control | delay | Significance | TFS) |
| 1 | 12 | 28.2 | — | — | — | 0/0/0 |
| 2 | 12 | 64.8 | 36.6 | 130 | — | 0/1/0 |
| 3 | 12 | 73.0 | 44.8 | 159 | ns | 0/0/0 |
| 4 | 12 | 73.4 | 45.2 | 160 | ns | 1/1/0 |
| 5 | 12 | 87.3 | 59.1 | 210 | ns | 1/0/0 |
| 6 | 12 | 78.7 | 50.5 | 179 | ns | 0/0/0 |

ns: P > 0.05;
*: 0.01 < P < 0.05;
**: 0.001 < P < 0.01;
***: P < 0.001

The combination of 2-(4-ethoxyphenyl)-4-methyl-1-(4-sulfamoylphenyl)-pyrrole and trastuzumab was well-tolerated. Treatment with 2-(4-ethoxyphenyl)-4-methyl-1-(4-sulfamoylphenyl)-pyrrole (10 mg/kg) and trastuzumab afforded a 210% Tumor growth delay.

### Example 11

### Treatment of Breast Cancer with a combination containing 2-(4-ethoxyphenyl)-4-methyl-1-(4-sulfamoylphenyl)-pyrrole along with trastuzumab and paclitaxel

A method for treating a patient having breast cancer comprising administering to the patient a therapeutically effective amount of a combination comprising 2-(4-ethoxyphenyl)-4-methyl-1-(4-sulfamoylphenyl)-pyrrole, trastuzumab and, optionally, an additional chemotherapy agent or their respective pharmaceutically acceptable salt, solvate or prodrug is contemplated. For women with metastatic breast cancer whose prior adjuvant treatment included anthracycline therapy, a treatment regimen comprising administration of 2-(4-ethoxyphenyl)-4-methyl-1-(4-sulfamoylphenyl)-pyrrole along with trastuzumab and paclitaxel is contemplated.

### Example 12

### Evaluation of 2(4-ethoxyphenyl)-4-methyl-1-(4-sulfamoylphenyl)-pyrrole and celecoxib as monotherapy

### MCF-7 Model:

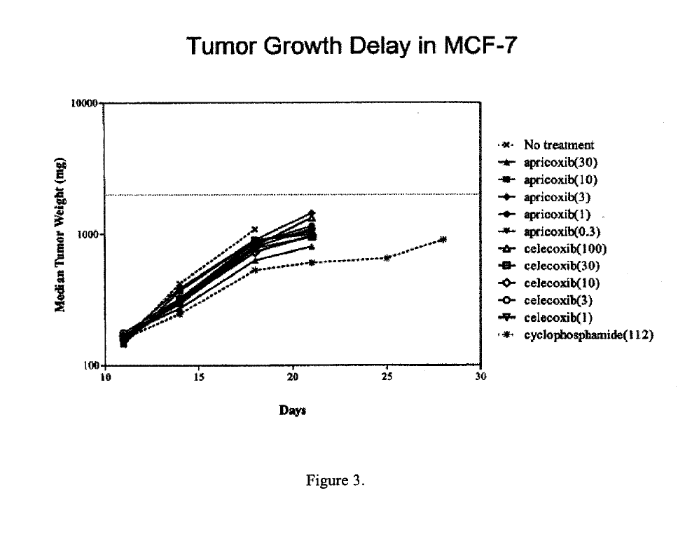

Female SCID mice (10 per group) were injected subcutaneously in the flank (1 mm³ MCF-7 tumor) and tumor volume was evaluated using a caliper twice a week for up to 90 days, longer for responders. Upon reaching an average tumor size of 100-200 mg the treatment was begun. Celecoxib and 2-(4-ethoxyphenyl)-4-methyl-1-(4-sulfamoylphenyl)-pyrrole were both formulated as a solution in 1% carboxymethyl cellulose/water and dosed orally by gavage. The endpoint for the study was when tumor volume reached 0.75 gms or 90 days, which ever came first. Mice injected with MCF-7 cancer cells were grouped into one of the following treatment groups:
Group 1: no treatment;
Group 2: cyclophosphamide 112 mg/kg Q4D×3;
Group 3: 2-(4-ethoxyphenyl)-4-methyl-1-(4-sulfamoylphenyl)-pyrrole (30 mg/kg, po, daily);
Group 4: 2-(4-ethoxyphenyl)-4-methyl-1-(4-sulfamoylphenyl)-pyrrole (10 mg/kg, po, daily);
Group 5: 2-(4-ethoxyphenyl)-4-methyl-1-(4-sulfamoylphenyl)-pyrrole (3 mg/kg, po, daily);
Group 6: 2-(4-ethoxyphenyl)-4-methyl-1-(4-sulfamoylphenyl)-pyrrole (1 mg/kg, po, daily);
Group 7: 2-(4-ethoxyphenyl)-4-methyl-1-(4-sulfamoylphenyl)-pyrrole (0.3 mg/kg, po, daily);
Group 8: celecoxib (100 mg/kg, po, daily);
Group 9: celecoxib (30 mg/kg, po, daily);
Group 10: celecoxib (10 mg/kg, po, daily);
Group 11: celecoxib (3 mg/kg, po, daily);
Group 12: celecoxib (1 mg/kg, po, daily)
   Response summary for MCF-7 study (see also FIG. 3)

| | | Median Total Time | Difference | Statistical |
|---|---|---|---|---|
| Group | n | to Endpoint (days) | from control | Significance |
| 1 | 10 | 5.5 | — | — |
| 2 | 10 | 14.7 | 9.2 | ns |
| 3 | 10 | 8.0 | 2.5 | ns |
| 4 | 10 | 7.3 | 1.8 | ns |
| 5 | 10 | 6.8 | 1.3 | ns |
| 6 | 10 | 7.1 | 1.6 | ns |
| 7 | 10 | 7.5 | 2.0 | ns |
| 8 | 10 | 7.1 | 1.6 | ns |
| 9 | 10 | 7.6 | 2.1 | ns |
| 10 | 10 | 7.6 | 2.1 | ns |
| 11 | 10 | 6.9 | 1.4 | ns |
| 12 | 10 | 7.2 | 1.7 | ns |

2-(4-ethoxyphenyl)-4-methyl-1-(4-sulfamoylphenyl)-pyrrole and celecoxib were well-tolerated. Neither 2-(4-ethoxyphenyl)-4-methyl-1-(4-sulfamoylphenyl)-pyrrole or celecoxib was active as a single agent in MCF-7.

1-111. (canceled)

112. A method for treating a subject having cancer, comprising administering to the subject, a therapeutically effective amount of a combination comprising a 1,2-diphenylpyrrole derivative and a HER2 [ErbB2] inhibitor or their respective pharmaceutically acceptable salt, solvate, polymorph or prodrug.

113. The method of claim 112 wherein the 1,2-diphenylpyrrole derivative has the following formula: 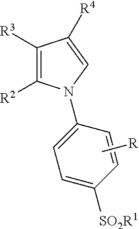
wherein:
R is a hydrogen atom, a halogen atom or an alkyl group having from 1 to 6 carbon atoms;
R¹ is an alkyl group having from 1 to 6 carbon atoms or an amino group;
R² is a phenyl group which is unsubstituted or is substituted by at least one substituent selected from the group consisting of substituents α and substituents β;
R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;
R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β;
said aralkyl group are an alkyl group having from 1 to 6 carbon atoms and which are substituted by at least one aryl group as defined above;
said substituents α are selected from the group consisting of a hydroxy group, a halogen atom, an alkoxy group having from 1 to 6 carbon atoms and an alkylthio group having from 1 to 6 carbon atoms;
said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

114. The method of claim 113 wherein:
R is a hydrogen atom, a halogen atom or an alkyl group having from 1 to 4 carbon atoms;
R¹ is a methyl group or an amino group;
R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;
R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

115. The method of claim 114 wherein:
R is a hydrogen atom;
R¹ is an amino group;
R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;
R³ is a hydrogen atom, a halogen atom, an alkyl group having from 1 to 4 carbon atoms or a haloalkyl group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

116. The method of claim 115 wherein the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

117. The method of claim 116 wherein the 1,2-diphenylpyrrole derivative is 2-(4-ethoxyphenyl)-4-methyl-1-(4-sulfamoylphenyl)-pyrrole.

118. The method of claim 112 wherein the inhibitor of HER2 [ErbB2] is selected from the group:

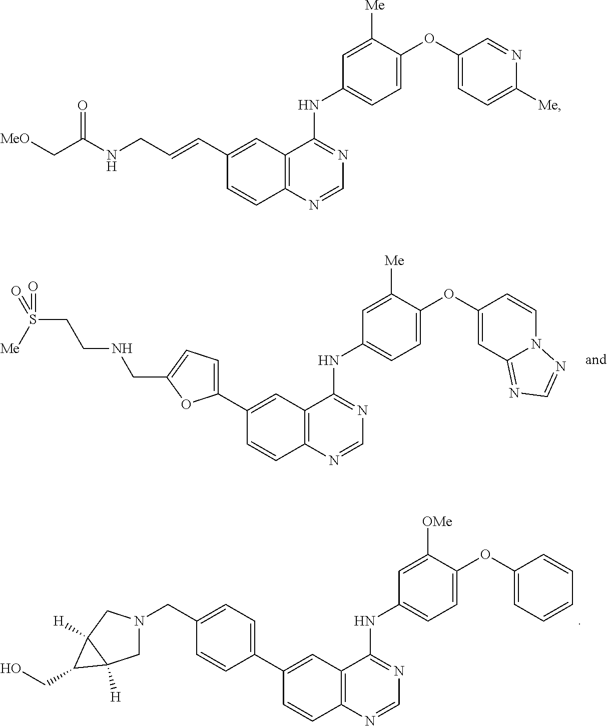

119. The method of claim 112 wherein the inhibitor of HER2 [ErbB2] is selected from ARRY-380, CP-724714 and CP-654577.

120. The method of claim 112 wherein the 1,2-diphenylpyrrole derivative is 2-(4-ethoxyphenyl)-4-methyl-1-(4-sulfamoylphenyl)-pyrrole and the inhibitor of HER2 [ErbB2] is selected from ARRY-380, CP-724714 and CP-654577.

121. The method of claim 112 wherein the 1,2-diphenylpyrrole derivative and the inhibitor of HER2 [ErbB2] are administered sequentially in either order or simultaneously.

122. The method of claim 112 wherein the 1,2-diphenylpyrrole derivative is administered first.

123. The method of claim 112 wherein the inhibitor of both EGFR [ErbB1] and HER2 [ErbB2] is administered first.

124. The method of claim 112 further comprising administering to the subject one or more therapies in addition to the combination of a 1,2-diphenylpyrrole derivative and an inhibitor of HER2 [ErbB2].

125. The method of claim 112 further comprising administering to the subject one or more therapies in addition to the combination comprising 2-(4-ethoxyphenyl)-4-methyl-1-(4-sulfamoylphenyl)-pyrrole and an inhibitor of HER2 [ErbB2] selected from ARRY-380, CP-724714 and CP-654577.

126. The method of claim 125 further comprising administering to the subject capecitabine in addition to the combination comprising 2-(4-ethoxyphenyl)-4-methyl-1-(4-sulfamoylphenyl)-pyrrole and an inhibitor of HER2 [ErbB2] selected from ARRY-380, CP-724714 and CP-654577.

127-208. (canceled)

209. The method of claim 126 wherein the cancer to be treated is cancer is selected from breast cancer, ovarian cancer, endometrial cancer, prostate cancer, gastric cancer, salivary gland cancer, pancreatic cancer, colorectal cancer, non-small cell lung cancers, oral cancers, and cutaneous squamous cell carcinoma.

210-211. (canceled)

212. The method of claim 126 further comprising administering to the subject one or more therapies in addition to the combination of 2-(4-ethoxyphenyl)-4-methyl-1-(4-sulfamoylphenyl)-pyrrole and an inhibitor of HER2 [ErbB2] selected from ARRY-380, CP-724714 and CP-654577.

213. The method of claim 212 wherein the one or more therapies comprise one or more of radiation therapy, chemotherapy, high dose chemotherapy with stem cell transplant; hormone therapy, and monoclonal antibody therapy.

214-217. (canceled)

218. The method of claim 213 wherein hormone therapy comprises administering to the subject tamoxifen, letrozole, anastrozole or exemestane.

219. The method of claim 213 wherein monoclonal antibody therapy comprises administering to the subject trastuzumab, trastuzumab-DM1 or pertuzumab.
